(19) United States
(12) Patent Application Publication
(10) Pub. No.: US 2022/0259386 A1
(43) Pub. Date: Aug. 18, 2022

(54) SUPRAMOLECULAR HYDROGELS

(71) Applicant: Technische Universiteit Eindhoven, EINDHOVEN (NL)

(72) Inventors: Mani DIBA, EINDHOVEN (NL); Sergio SPAANS, EINDHOVEN (NL); Patricia Yvonne Wilhelmina DANKERS, EINDHOVEN (NL)

(21) Appl. No.: 17/626,373

(22) PCT Filed: Jul. 13, 2020
(86) PCT No.: PCT/EP2020/069787

§ 371 Date: Jan. 11, 2022

Related U.S. Application Data
(60) Provisional application No. 62/873,449, filed on Jul. 12, 2019.

(51) Int. Cl. C08J 3/075; C08G 65/333; C08G 83/00; C08G 18/48; C08G 18/10; C08G 18/73; C12N 5/00; C12N 5/071; A61K 9/06; A61K 31/785

(52) U.S. Cl. CPC C08J 3/075; C08G 65/33396; C08G 83/008; C08G 18/4833; C08G 18/10; C08G 2210/00; C12N 5/0018; C12N 5/067; A61K 9/06; A61K 31/785; C08J 2371/02; C08G 18/73

(57) ABSTRACT

The present invention relates to a method of producing a supramolecular hydrogel which is formed by the mixing and gelation of at least two dispersions of different types of synthetic hydrogelators, said hydrogelators being formed of synthetic building blocks comprising one or more hydrogen bonding units, wherein each bonding unit comprises a ureido-pyrimidinone subunit and each bonding unit is conjugated with a hydrophilic polymer unit, the method comprising the steps of: a) providing a first dispersion of one type of hydrogelators, b) mixing the first dispersion with a second dispersion of another type of hydrogelators, and c) allowing the dispersions to form the hydrogel, wherein the types of hydrogelators are selected from multifunctional hydrogelators and monofunctional hydrogelators, wherein the steps of the method are conducted under biocompatible conditions, and wherein the hydrophilic polymer unit of the hydrogelators comprised in the first dispersion has a minimal hydrophilicity such that the first dispersion does not form a hydrogel under the biocompatible conditions applied.

### TECHNICAL FIELD

The present invention relates to a method of producing a supramolecular hydrogel. The invention further relates to a supramolecular hydrogel obtainable by the method of the present invention. The present invention also relates to the in vitro and in vivo use of the supramolecular hydrogel obtainable by the method of the present invention and a kit for producing a supramolecular hydrogel of the present invention.

### BACKGROUND

Molecular self-assembly is a ubiquitous process in nature, and is also believed to play an essential role in the emergence, maintenance, and advancement of life. While the primary focus of the research on molecular self-assembly focuses on the bio-macromolecules (proteins, nucleic acids, and polysaccharides) or their mimics, the self-assembly of small molecules in water (or an organic solvent) also has profound implications from fundamental science to practical applications. Because one usual consequence of the self-assembly of the small molecules is the formation of a gel (or gelation), a subset of these small molecules is called gelators. Depending on the solvents in which they form gels, these small molecules are further classified as hydrogelators (using water as the liquid phase) and organo-gelators (using an organic “solvent” as the liquid phase). More precisely, hydrogelators (i.e., the molecules) self-assemble in water to form three-dimensional supramolecular networks that encapsulate a large amount of water to afford an aqueous mixture. The aqueous mixture is a supramolecular hydrogel because it exhibits viscoelastic behaviour of a gel (e.g. unable to flow without shear force). Unlike the conventional polymeric hydrogels that are mainly based on covalently cross-linked networks of polymers (i.e. gellant), the networks in supramolecular hydrogels are formed due to noncovalent interactions between the hydrogelators.

Because supramolecular hydrogels are a type of relatively simple heterogeneous system that consists of a large amount of water, it is not surprising that the applications of hydrogels and hydrogelators in life science have advanced most significantly. Water-soluble supramolecular hydrogels constitute an attractive class of hydrogels, since the non-covalent interactions between the molecular components can result in tuneable hydrogels with a highly dynamic structure. When applied as a biomaterial, the dynamic, adaptable, and reversible interactions between the hydrogel and embedded (stem) cells are proposed to closely mimic systems and processes found in nature. The artificial biomaterial developed shall not only contain specific mechanical properties, but also has to present proteins and peptides to cells in a reversible, adaptive and spatiotemporal manner in order to regulate cell response. Therefore, control over the structural and dynamic properties of hydrogels is important for the design and development of supramolecular biomaterials, which can only be achieved when there is profound understanding at the molecular level.

### DESCRIPTION

In order to provide an artificial biomaterial the invention provides hereto a method of producing a supramolecular hydrogel, wherein the supramolecular hydrogel is formed by the mixing and gelation of at least two dispersions of different types of synthetic hydrogelators. The synthetic hydrogelators are formed of synthetic building blocks comprising one or more hydrogen bonding units, wherein each bonding unit comprises a ureido-pyrimidinone subunit and each bonding unit is conjugated with a hydrophilic polymer unit. The method comprises the steps of:

a) providing a first dispersion of one type of synthetic hydrogelators;

b) mixing the first dispersion of one type of synthetic hydrogelators provided in step a) with a second dispersion of another type of synthetic hydrogelators; and

c) allowing the dispersions of synthetic hydrogelators mixed in step b) to form the supramolecular hydrogel,

wherein the types of synthetic hydrogelators are selected from the group consisting of multifunctional synthetic hydrogelators comprising two or more hydrogen bonding units and monofunctional synthetic hydrogelators comprising one hydrogen bonding unit. In the method of the present invention the steps of the method are conducted under biocompatible conditions. In order to provide the artificial biomaterial the hydrophilic polymer unit of the synthetic hydrogelators comprised in the first dispersion has a minimal hydrophilicity such that the first dispersion does not form a hydrogel under the biocompatible conditions applied to the method. By providing dispersions of synthetic hydrogelators wherein at least one dispersion does not form a hydrogel under biocompatible conditions, the formation of an hydrogel at biocompatible conditions upon mixing of the at least one dispersion of synthetic hydrogelators with at least one other dispersion of synthetic hydrogelators is herewith provided. Due to the fact that the method of the present invention is performed under biocompatible conditions, the method thus allowing biological material to be seeded (i.e. added to) and/or formed in the at least one dispersion before gelation of the hydrogel. In other words, gelation of the hydrogel of the present invention is not effectuated by altering, i.e. changing the conditions such as pH or temperature, the biocompatible conditions, but by mixing different dispersions of synthetic hydrogelators. Optionally, the mixture of dispersions of synthetic hydrogelators may be activated to form a hydrogel by adding a gelating agent.

Given the above, it is stressed that the first and the second dispersion may comprise each type of hydrogelator. So, in case the synthetic hydrogelators of the first dispersion are selected from the group consisting of multifunctional synthetic hydrogelators, the synthetic hydrogelators of the second dispersion are selected from the group consisting of monofunctional synthetic hydrogelators. Also, in case the synthetic hydrogelators of the first dispersion are selected from the group consisting of monofunctional synthetic hydrogelators, the synthetic hydrogelators of the second dispersion are selected from the group consisting of multifunctional synthetic hydrogelators. It is further noted that the ‘type of hydrogelators’ refers to the number of hydrogen bonding units. In other words, a dispersion comprising one type of hydrogelators being monofunctional synthetic hydrogelators, does mean that the dispersion comprises identical or different molecules/configurations of monofunctional synthetic hydrogelators.

As used herein, the term “dispersion” refers to a continuous liquid medium containing a suspension of minute particles or aggregates. The liquid medium is preferably an aqueous medium, such as water or a biocompatible aqueous medium, such as saline or cell culture medium. The dispersions of the present invention may be referred to as solutions, e.g. aqueous solutions, comprising synthetic hydrogelators. In this respect it is noted that the hydrogelators of the present invention may be molecular dissolved (i.e. being in solution) or reside as self-assembled molecular aggregates that do not form crosslinks and therefore display the behaviour of a solution. The non-formation of crosslinks results in a solution/dispersion that does not display the behaviour of a hydrogel.

A particular advantage of the present invention is that the biocompatible conditions are not altered during the process of forming a hydrogel. By providing a method wherein the biocompatible conditions are met throughout the process, any biological material present during the formation of the hydrogel, such as cells, spheroids and/or organoids, is not harmed or damaged by changing conditions, including pH and temperature.

As used herein, the term “biocompatible conditions” refers to conditions that does not harm or damage any bioactive material present in the hydrogel and/or hydrogel to be formed. Other suitable terms for the term “biocompatible” may include “cell-compatible” or “cyto-compatible”. The term “biocompatible conditions”, “cell-compatible conditions” or “cyto-compatible conditions” preferably includes conditions wherein biological material is not harmed or damaged. Most important parameters include temperature and pH. The pH of the conditions used throughout the method of the present is preferably neutral, i.e. a pH of about 6 to 8. More preferably the pH is a physiological pH of about 7.4. With regard to the temperature, in order to meet the conditions of the method of the present invention, i.e. being cell-compatible, the temperature does not exceed 40° C. Preferably, the temperature is within the range of about 4° C. to about 40° C., more preferably about 10° C. to 38° C., even more preferably about 20° C. to about 37.5° C. Other preferred temperature ranges are selected from a physiological temperature of about 36.5° C. to about 37.5° C. or a more convenient temperature such as room temperature. It is note that other parameters may also have some influence on the conditions of a bioactive material, e.g. pressure, ionic strength and the like.

In an embodiment of the present invention, the dispersions of synthetic hydrogelators may be prepared under biocompatible conditions as well. Although the dispersions may be prepared under different conditions, e.g. including conditions that are not biocompatible, the preparation of the dispersions of synthetic hydrogelators under cell-compatible conditions is preferred. It is noted that conditions that are not biocompatible, e.g. high temperatures or high pH, can be used during the preparation of the dispersions. Still, the preparation of biocompatible dispersions under non-biocompatible conditions, may result in biocompatible dispersions as long as the dispersions brought under biocompatible conditions before further use, e.g. prior to mixing the dispersions and/or adding biological material to the dispersion.

As used herein, the term “biological material” refers to either human or non-human material and refers to any substance derived or obtained from a living organism (including plant material). Illustrative examples of biological materials include, but are not limited to, the following: cells, tissues, spheroids, organoids, blood or blood components, proteins, DNA, and the like.

As used herein, the term “synthetic hydrogelators” refers to synthetic compounds that are capable of forming a hydrogel. The hydrogelators of the present invention are designed such that they form ureido-pyrimidinone supramolecular complexes. The hydrogelators of the present invention comprise at least one ureido-pyrimidinone (also referred to as “UPy”) comprising hydrogen bonding unit. The present invention makes a distinction between synthetic hydrogelators functionalized with one ureido-pyrimidinone hydrogen bonding unit and synthetic hydrogelators functionalized with two or more ureido-pyrimidinone hydrogen bonding units.

As used herein, the term “monofunctional synthetic hydrogelators” refers to hydrogelators functionalized with one ureido-pyrimidinone hydrogen bonding unit. The monofunctional synthetic hydrogelators typically comprise a hydrophilic polymer unit (also referred to as a “tail unit”) of which one end of the polymer unit is conjugated, e.g. covalently bound or linked, to the hydrogen bonding unit. The other end of the polymer unit (also referred to as a “free end”) is preferably conjugated to a hydrophilic end unit or functional subunit, such as a bioactive subunit.

As used herein, the term “multifunctional synthetic hydrogelators” refers to hydrogelators functionalized with two or more ureido-pyrimidinone hydrogen bonding units. The multifunctional synthetic hydrogelators typically comprise one or more hydrophilic polymer units (also referred to as “spacer units”) conjugated, e.g. covalently bound or linked, to the hydrogen bonding units. The number of hydrogen bonding units present in the hydrogelator molecule is reflected by the prefix used, i.e. multifunctional synthetic hydrogelators functionalized with two hydrogen bonding units are herein referred to as “bifunctional synthetic hydrogelators”. Hydrogelators functionalized with three hydrogen bonding units are herein referred to as “trifunctional synthetic hydrogelators” and so on. With regard to the polymer units, it is noted that both ends of the polymer unit may be conjugated to a hydrogen bonding unit. Although a linear hydrogelator compound is preferred wherein the hydrogen bonding units are linked via polymer units in series, other arrangements or designs of hydrogelator compounds may be suitable as well. For example, a ‘star’-like compound may be designed, wherein one end of each polymer unit is conjugated to a hydrogen bonding unit, and wherein the other free ends of the polymer units are linked to each other.

As used herein, the term “supramolecular complex” is a complex made of assembled hydrogelators. The forces responsible for the spatial organization may vary from weak (intermolecular forces, electrostatic bonding) to strong (hydrogen bonding), provided that the degree of electronic coupling between the molecular component remains small with respect to relevant energy parameters of the component. A supramolecular complex is different from a chemical complex in that in a supramolecular complex the interactions between subunits of the hydrogelators are mainly the weaker and reversible non-covalent interactions between molecules, whereas in traditional chemistry the interactions are covalent. These interactions in supramolecular complexes include hydrogen bonding, metal coordination, hydrophobic forces, van der Waals forces, pi-pi interactions and electrostatic effects. Important concepts that are indicative of supramolecular chemistry include molecular self-assembly, folding, molecular recognition, host-guest chemistry, mechanically-interlocked molecular architectures, and dynamic covalent chemistry. For the purpose of the present invention, the subunits form a supramolecular complex by self-assembly and the forces holding the subunits together are preferably hydrogen bonding.

The supramolecular complexes of the present invention form a “supramolecular hydrogel” as used herein to describe the resulting product as a result of the gelation of the hydrogelators used. The gelation, or formation of a hydrogel, is a result of the self-assembly or cross-linking of the hydrogelators used. Also, with regard to the present invention, the phrase “does not form a hydrogel” as used herein refers to the situation that the hydrogelators present in the dispersion does not self-assemble or does not gelate to form supramolecular complexes, i.e. a hydrogel.

It is further noted that the phrase “does not form a hydrogel”, i.e. the hydrogelators present in the dispersion do not gelate in order to form a hydrogel has to be interpreted that the behaviour of the dispersion of synthetic hydrogelators does not behave like a hydrogel before mixing both dispersions. It was found that before mixing the dispersions of synthetic hydrogelators self-assembling behaviour of the synthetic hydrogelators resulting in molecular aggregates was observed before mixing the dispersions. However, it was also found that under the biocompatible conditions chosen the self-assemblies or aggregates of synthetic hydrogelators do not behave like a hydrogel, i.e. do not form supramolecular complexes forming a hydrogel. Upon mixing the aggregates or self-assemblies of synthetic hydrogelators, interacting, crosslinking, non-covalent binding between the aggregates or self-assemblies of synthetic hydrogelators occurs, such that the mixed dispersions do display hydrogel behaviour. In other words, self-assembly of the synthetic hydrogelators according to the present invention may occur under all conditions, whereas given the types and mixing of hydrogelators of the present invention hydrogel behaviour only occurs after mixing the at least two dispersions of different types of synthetic hydrogelators.

As used herein, the term “bioactive supramolecular hydrogel” refers to a hydrogel formed of supramolecular complexes and a liquid, preferably an aqueous liquid, such as water, wherein the hydrogel is capable of stimulating, directing or eliciting a specific biological response at the interface of the material, which may result in, for example, the formation of a bond between tissue and said bioactive material.

Although the synthetic hydrogelators used in the method of the present invention may be selected from the group consisting of any hydrogelator functionalized with at least one ureido-pyrimidinone hydrogen bonding unit, preferred hydrogelators are described in, for example, International patent application published under WO 2016/028149 A1. However, it is stressed that other designs of hydrogelators may be used as well. For example, FIG. 1 discloses four supramolecular molecules as used in the method of the present invention including:
a bifunctional synthetic hydrogelator comprising two ureido-pyrimidinone subunits as hydrogen bonding units each conjugated to a polyethylene glycol polymer unit via a hexyl-urea-dihexyl subunit, herein referred to as “UPy-PEG-UPy”;
a monofunctional synthetic hydrogelator comprising one ureido-pyrimidinone subunit as hydrogen bonding unit conjugated to an oligo ethylene glycol polymer unit via a hexyl-urea-dihexyl subunit, wherein the free end of the polymer unit comprises glycine, herein referred to as “UPy-OEG-Glycine” or “UPy-OEG-G”;
a monofunctional synthetic hydrogelator comprising one ureido-pyrimidinone subunit as hydrogen bonding unit conjugated to an oligo ethylene glycol polymer unit via a hexyl-urea-dihexyl subunit, wherein the free end of the polymer unit comprises a Cy5 dye, herein referred to as “UPy-OEG-Cy5”; and
a monofunctional synthetic hydrogelator comprising one ureido-pyrimidinone subunit as hydrogen bonding unit conjugated to an oligo ethylene glycol polymer unit via a hexyl-urea-dihexyl subunit, wherein the free end of the polymer unit comprises a cyclic RGD, herein referred to as “UPy-OEG-cRGD”.

As it can be derived from FIG. 1, the hydrogen bonding units of the synthetic hydrogelators as used in both dispersions are identical. Although the method of the present invention may be performed wherein the hydrogen bonding units of the synthetic hydrogelators comprised in the at least two dispersions of synthetic hydrogelators are different, it is preferred that the hydrogen bonding units of the synthetic hydrogelators comprised in the at least two dispersions of synthetic hydrogelators are identical. By providing dispersions comprising synthetic hydrogelators having identical hydrogen bonding units, the assembly of the supramolecular complexes to form a hydrogel can be facilitated in a controllable and reliable manner. In other words, by providing dispersions comprising synthetic hydrogelators having identical hydrogen bonding units, the hydrogelators are easier stackable than dispersions comprising synthetic hydrogelators having different hydrogen bonding units.

It is however submitted that the hydrophilicity of the polymer unit is selected such that the hydrogelators of the at least first dispersion, but alternatively also the second dispersion, do not form an hydrogel under the conditions that are applicable to the method of the present invention. It was found that the hydrophilicity should be chosen such that the hydrophilicity of the polymer unit is not too low (too hydrophobic) but also not too high. In a preferred range the hydrophilicity of the polymer unit comprised in the synthetic hydrogelator of the present invention correspond to the hydrophilicity of a monodisperse polyethylene glycol having 5 to 50 oxyethylene units. Preferably the hydrophilicity is selected such that the hydrophilicity corresponds to the hydrophilicity of a monodisperse polyethylene glycol having 7 to 40 oxyethylene units, preferably having 9 to 30 oxyethylene units, more preferably having 11 to 20 oxyethylene units.

In an embodiment of the present invention, besides having a first dispersion of synthetic hydrogelators that does not form a hydrogel under the biocompatible conditions applied to the method, the hydrophilic polymer unit of the synthetic hydrogelators comprised in the second dispersion has a minimal hydrophilicity such that the second dispersion does not form a hydrogel under the biocompatible conditions applied to the method. By also providing a second dispersion that can be prepared and maintained under biocompatible conditions, the bio- or cell-compatibility of the second dispersion is further enhanced.

Further to the ureido-pyrimidinone subunit, the hydrogen bonding units may further comprises an urea subunit and/or urethane subunit linking the ureido-pyrimidinone subunit with the hydrophilic polymer unit of the respective monofunctional synthetic hydrogelator and multifunctional synthetic hydrogelator.

In a further embodiment of the present invention, the method may be seeded with biological material, such as cells, spheroids and/or organoids. Preferably, the method of the present invention comprises the step of:
before mixing the first dispersion of synthetic hydrogelators with the second dispersion of synthetic hydrogelators in step b), adding biological material, such as cells, spheroids and/or organoids, to the first dispersion of synthetic hydrogelators.

Alternatively, in addition to the above-defined seeding step, the method may further comprise the step: adding biological material to the second dispersion of synthetic hydrogelators before mixing the first and second dispersions to form the hydrogel of the present invention.

It was found that by mixing the biological material with the first dispersion comprised of, preferably multifunctional synthetic hydrogelators, before mixing the at least two dispersions, the a reliable and reciprocal method can be provided wherein the cell behaviour is reliably directed in the desired way.

In step b), the first and second dispersions of synthetic hydrogelators may be mixed such that the molar ratio between the multifunctional synthetic hydrogelators and monofunctional synthetic hydrogelators is at least 1:1, preferably at least 1:10, more preferably at least 1:50. A preferred range of molar ratio between the multifunctional synthetic hydrogelators and monofunctional synthetic hydrogelators is about 1:1-150, preferably about 1:50-125, more preferably about 1:75-100.

The total amount of synthetic hydrogelators in step b) is preferably at most 25 wt.-% of the total weight of the dispersions mixed. More preferably the total amount of synthetic hydrogelators in step b) is between 0.5 wt.-% and 20 wt.-%, even more preferably between 1.5 wt.-% and 10 wt.-%, between 2.0 wt-% and 5 wt-%, and most preferably about 2.5 wt-% or about 5.0 wt.-%.

The multifunctional synthetic hydrogelators may be preferably selected from the group consisting of bifunctional synthetic hydrogelators comprising two hydrogen bonding units.

With regard to the monofunctional synthetic hydrogelators it is noted that the hydrophilic polymer unit may be provided, preferably at its free end opposite the end conjugated to the hydrogen bonding unit, with a functional subunit, such as a bioactive subunit. Such bioactive subunit may include a bioactive feature directing cell behaviour, such as cell growth, cell adhesion, cell spreading, cell migration, cell differentiation and combinations thereof and/or a bioactive feature having antimicrobial activity. By providing a monofunctional synthetic hydrogelator having a bioactive unit, the bioactivity of the supramolecular hydrogel to be formed may be altered or directed to have a desired property. Besides using a bioactive subunit, the hydrophilic polymer unit of the monofunctional synthetic hydrogelators may comprise at one end, which one end is not conjugated to the hydrogen bonding unit, a functional subunit useful for use in labelling and imaging techniques.

Examples of useful functional groups may include:
ECM-derived peptides: RGD, DGEA, YIGSR, PHSRN (as described by: Dankers, et al. Bioengineering of living renal membranes consisting of hierarchical, bioactive supramolecular meshes and human tubular cells, in Biomaterials 2011, 32, 723-733; and Feijter, de et al. Solid-phase based synthesis of ureidopyrimidinone-peptide conjugates for supramolecular biomaterials, in Synth. Lett. 2015, 26, 2707-2713);
Collagen binding peptide: HVWMQAP and ECM-derived peptides: RGD, PHSRN (as described by: Wisse, et al. Multicomponent supramolecular thermoplastic elastomer with peptide-modified nanofibers, in J. Pol.

Sci. Part A 2011, 49, 1764-1771; and Kieltyka, et al. Modular synthesis of supramolecular ureidopyrimidinone-peptide conjugates using an oxime ligation strategy, in Chem. Commun. 2012, 48, 1452-1454);
Peptide as co-factor for enzyme (S-peptide) (as described by: Appel, et al. Enzymatic activity at the surface of biomaterials via supramolecular anchoring of peptides: the effect of material processing, in Macromolec. Biosci. 2011, 11, 1706-1712);
ECM-derived peptide: RGD (as described by: Mollet, et al. A modular approach to easily processable supramolecular bilayered scaffolds with tailorable properties, in J. Mater. Chem. B. 2014, 2, 2483-2493; and Gaal, van et al. Functional peptide presentation on different hydrogen bonding biomaterials using supramolecular additives, in Biomaterials 2019, doi: 10.1016/j.biomaterials.2019.119466);
SDF1alpha (=chemokine) truncated peptides: SKPVVLSYR, SKPVSLSYR (as described by: Muylaert, et al. Early in-situ cellularization of a supramolecular vascular graft is modified by synthetic stromal cell-derived factor-1α derived peptides, in Biomaterials 2016, 76, 187-195);
Heparin binding peptide: GLRKKLGKA (as described by: Bonito, et al. Modulation of macrophage phenotype and protein secretion via heparin-IL-4 functionalized supramolecular elastomers, in Acta Biomater. 2018, 71, 247-260);
Anti-microbial peptides (long peptide sequences) (as described by: Zaccaria, et al. Antimicrobial peptide modification of biomaterials using supramolecular additives, in J. Pol, Sci. Part A Polym. Chem. 2018, doi: 10.1002/pola.29078);
Protein G—binding to Fc domain (protein; antibodies) (as described by: Putti, et al. A supramolecular platform for the introduction of Fc-fusion bioactive proteins on biomaterial surfaces, ACS Applied Polymer Materials 2019, 1, 2044-2054);
Reactive groups for click-chemistry: tetrazine (as described by: Goor, et al. Efficient functionalization of additives at supramolecular material surfaces, in Adv. Mater. 2017, 29, 1604652; and Goor, et al. Introduction of anti-fouling coatings at the surface of supramolecular elastomeric materials via post-modification of reactive supramolecular additives, in Polym. Chem. 2017, 8, 5228-5238);
Reactive groups: catechol (as described by: Spaans, et al. Supramolecular surface functionalization via catechols for the improvement of cell-material interactions, in Biomater. Sci. 2017, 5, 1541-1548) and
Initiator for polymerization (as described by: Ippel, et al Supramolecular additive-initiated controlled atom transfer radical polymerization of zwitterionic polymers on ureido-pyrimidinone-based biomaterial surfaces, in Macromolecules 2020, doi: 10.1021/acs.macromol.0c00160).

Examples of other useful functional groups may include imaging labels such as:
Growth factor stabilizing sulfonated peptides (long peptide sequence) (in solution/dispersion; and in hydrogel) (as described by: Hendrikse, et al. A supramolecular platform stabilizing growth factors, in Biomacromolecules 2018, 19, 2610-1617);
Notch-signaling peptide, Jagged1 (long peptide sequence) (in solution/dispersion) (as described by: Putti, et al. Influence of the assembly state on the functionality of a supramolecular Jagged1-mimicking peptide additive, in ACS Omega 2019, 4, 8178-8187);
Fluorophores: Cy3, Cy5 (in solution/dispersion) (as described by: Hendrikse, et al. Controlling and tuning the dynamic nature of supramolecular polymers in aqueous solutions, in Chem. Comm. 2017, 53, 2279-2282); and
MRI-Label: DOTA-Gd (in hydrogel) (as described by: Bakker, et al. MRI visualization of injectable ureidopyrimidinone hydrogelators by supramolecular contrast agent labeling, Adv. Healthcare Mater. 2018, doi: 10.1002/adhm.201701139).

The method of the present invention may further comprises the steps of:

d) after formation of the supramolecular hydrogel, culturing biological material, such as cells, spheroids and/or organoids, for a period of time; and

e) optionally, removing the hydrogel by using external stimuli.

Removal of the hydrogel may be performed by gentle mechanical disruption of the hydrogel, or using enzymatic or UV based stimuli dissolving the hydrogel, without dissolving the biological material encapsulated, included or present in the hydrogel.

The present invention also relates to a supramolecular hydrogel obtainable by the method of the present invention. In a preferred embodiment of the present invention, the supramolecular hydrogel is a bioactive supramolecular hydrogel. However, also non-bioactive supramolecular hydrogels may be of particular use. Such non-bioactive supramolecular hydrogel may be used for providing a carrier matrix encapsulating biological material, such as cells, organoids or spheroids, for the (local) delivery of the biological material without interacting with the network formed by the supramolecular complex of the hydrogel.

The hydrogel of the present invention may be used in parenteral, topical and sprayable applications. Further the hydrogel of the present invention may be used in a preform application for implantation into the human or animal body.

The present invention further relates to the in vitro use of the supramolecular hydrogel obtainable by the method of the present invention, directing of cell behaviour, such as cell growth, cell adhesion, cell spreading, cell migration, cell differentiation and combinations thereof.

In another aspect of the present invention, the invention relates to a supramolecular hydrogel obtainable by the method of the present invention for in vivo application, such as tissue or organ regeneration or therapies.

The present invention further relates to a kit for producing a supramolecular hydrogel, wherein the kit comprises at least two dispersions of synthetic hydrogelators, wherein the at least two dispersions of different types of synthetic hydrogelators comprise a first dispersion of one type of synthetic hydrogelators and a second dispersion of another type of synthetic hydrogelators for use in the method of the present invention. The kit of the present invention may further comprise components for preparing the dispersions of synthetic hydrogelators.

As already noted above, the different types of synthetic hydrogelators are selected from the group consisting of multifunctional synthetic hydrogelators comprising two or more hydrogen bonding units and monofunctional synthetic hydrogelators comprising one hydrogen bonding unit.

In an embodiment of the present invention, the dispersions may comprise different hydrogelators being of the same type of hydrogelators. In other words, the dispersions may comprise various different monofunctional synthetic hydrogelators or various different multifunctional synthetic hydrogelators (depending on the type of hydrogelators comprised in the dispersion) within the same dispersion. In fact, functionality of the dispersion is altered by adding or replacing an predefined amount of one type of a synthetic hydrogelator with a functionalized hydrogelator of the same type.

As a closing remark, it is submitted that the above method and hydrogel overcome some of the challenges experienced in the field of making supramolecular hydrogels, i.e. the technical challenges for (biomedical) applications using supramolecular hydrogels:
enabling effective/functional incorporation of bioactive motifs;
enabling easy removal/extraction of cells and multicellular structures from hydrogel matrix;
enabling adjustable degradation of hydrogels;
enabling adjustable and controlled release of compounds;
enabling controlled sol-to-gel transition resulting in easy injectability at physiological pH and temperature; and
enabling the control of the mechanical properties.
   By providing the method of the present invention, the above challenges are overcome in a simple, robust and reliable manner.

Embodiments of the invention could be a fully synthetic hydrogel matrix for cell biological and therapeutic applications, including in vitro, in vivo (including in situ), and clinical applications.

Embodiments of the invention can be applied in areas including cell or drug delivery systems, as a fully synthetic hydrogel matrix for cell biological and therapeutic applications, including in vitro, in vivo, and clinical applications.

### EXAMPLES

### Synthesis of UPy-PEG-UPy

UPy-PEG-UPy molecules were synthesized as described by Dankers, et al. Hierarchical Formation of Supramolecular Transient Networks in Water: A Modular Injectable Delivery System (Advanced Materials 2012, 24 (20): 2703-2709).

### Synthesis of UPy-OEG-cRGD and UPy-OEG-G

All reagents, chemicals, materials and solvents were obtained from commercial sources and were used as received. All solvents were of AR quality. In the synthetic procedures, equivalents (eq) are molar equivalents. ¹H-NMR spectra were recorded on a Bruker Avance III HD spectrometer at 298 K (400 MHz for ¹H-NMR). Chemical shifts are reported in ppm downfield from TMS at room temperature. Abbreviations used for splitting patterns are s=singlet, t=triplet, q=quartet, m=multiplet and br=broad. HPLC-PDA/MS was performed using a Shimadzu LC-10 AD VP series HPLC coupled to a diode array detector (Finnigan Surveyor PDA Plus detector, Thermo Electron Corporation) and an Ion-Trap (LCQ Fleet, Thermo Scientific). HPLC-analyses were performed using a Alltech Alltima HP C₁₈ 3μ column using an injection volume of 1-4 μL, a flow rate of 0.2 mL min⁻¹ and typically a gradient (5% to 100% in 10 min, held at 100% for a further 3 min) of MeCN in H₂O (both containing 0.1% formic acid) at 298 K. Preparative RP-HPLC (MeCN/H₂O with 0.1 v/v % formic acid) was performed using a Shimadzu SCL-10A VP coupled to two Shimadzu LC-8A pumps and a Shimadzu SPD-10AV VP UV-vis detector on a Phenomenex Gemini 5μ 018 110A column.

The route of synthesis of the UPy-OEG-cRGD (3) and UPy-OEG-G (4) is depicted below. Starting material 1 was synthesized as described by de Feijter, et al. Solid-Phase-Based Synthesis of Ureidopyrimidinone-Peptide Conjugates for Supramolecular Biomaterials (Synlett 2015, 26 (19): 2707-2713)

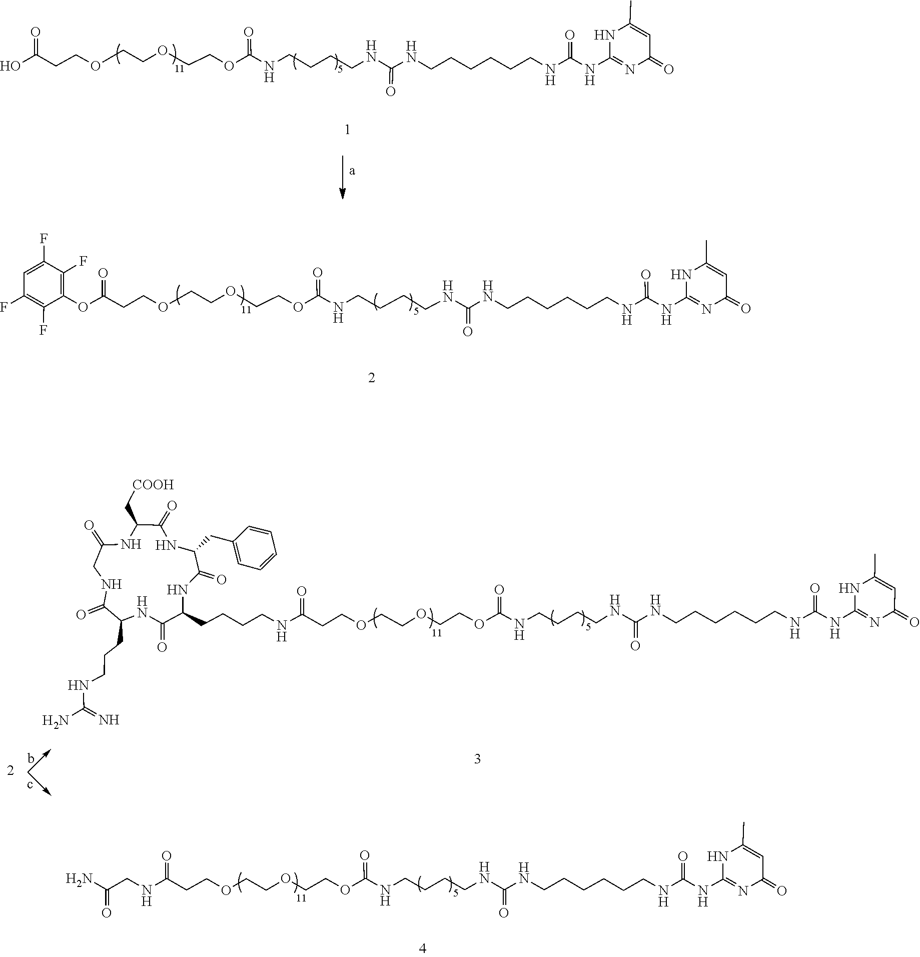

where:
(a) is 2,3,5,6-Tetrafluorophenol, pyridinium-p-toluenesulfonate, 1-ethyl-3-(3-dimethylaminopropyl)carbodiimide hydrochloride, CHCl₃, r.t., 2 h, 100%;
(b) is c(RGDfK), N,N-diisopropylethylamine, DMF, r.t., 1 h, 70%; and
(c) is glycinamide hydrochloride, N,N-diisopropylethylamine, CHCl₃/DMF 1:2, r.t., 1 h, 93%.

### Synthesis of 2,3,5,6-Tetrafluorophenyl 1-((6-methyl-4-oxo-1,4-dihydropyrimidin-2-yl)amino)-1,10,25-trioxo-26,29,32,35,38,41,44,47,50,53,56,59,62-tridecaoxa-2,9,11,24-tetraazapentahexacontan-65-oate (2)

1-((6-Methyl-4-oxo-1,4-dihydropyrimidin-2-yl)amino)-1,10,25-trioxo-26,29,32,35,38,41,44,47,50,53,56,59,62-tridecaoxa-2,9,11,24-tetraazapentahexacontan-65-oic acid (1) (41 mg, 36 μmol), 2,3,5,6-tetrafluorophenol (12 mg, 70 μmol, 2 eq) and pyridinium-p-toluenesulfonate (1.0 mg, 4 μmol, 0.1 eq) were dissolved in CHCl₃ (500 μL). 1-Ethyl-3-(3-dimethylaminopropyl)carbodiimide hydrochloride (14 mg, 72 μmol, 2 eq) was added and the solution was stirred at room temperature for 2 h. CHCl₃ (50 mL) was added and the solution was washed with water (3×20 mL). The combined organic layers were dried using Na₂SO₄ and filtrated. Evaporation of the solvent in vacuo yielded pure 2 (46 mg, 36 μmol, 100%) as a colorless solid. ¹H-NMR (CDCl₃): δ=13.16 (br s, 1H, NH, UPy), 11.82 (br s, 1H, NH, UPy), 10.07 (br s, 1H, NH, UPy), 7.01 (m, 1H, ArH), 5.83 (s, 1H, C═CH, UPy), 4.91 (br t, 1H, NH), 4.83 (br t, 1H, NH), 4.63 (br t, 1H, NH), 4.20 (t, 2H, NHC(O)OCH₂), 3.89 (t, 2H, NHC(O)OCH₂CH₂), 3.71-3.56 (m, 46H, OCH₂), 3.24 (q, 2H, CH₂NHC(O)O), 3.15 (m, 6H, CH₂NHC(O)NH), 2.96 (t, 2H, CH2C(O)O), 2.24 (s, 3H, CH₃), 1.64-1.20 (m, 28H, CH₂CH₂CH₂). ESI-MS: m/z Calc. for C₅₉H₉₉F₄N₇O₁₉ 1285.69; Obs. [M+2H]²⁺ 643.92, [M+H]⁺ 1286.42, [M+Na]⁺ 1308.42.

### Synthesis of UPy-OEG-cRGD (3): 2-((2S,5R,8S,11S)-5-Benzyl-11-(3-guanidinopropyl)-8-(1-((6-methyl-4-oxo-1,4-dihydropyrimidin-2-yl)amino)-1,10,25,65-tetraoxo-26,29,32,35,38,41,44,47,50,53,56,59,62-tridecaoxa-2,9,11,24,66-pentaazaheptacontan-70-yl)-3,6,9,12,15-pentaoxo-1,4,7,10,13-pentaazacyclopentadecan-2-yl)Acetic Acid

A solution of 2 (113 mg, 88 μmol) in DMF (1.4 mL) was added to a stirring solution of c(RGDfK) (double TFA-salt, 103 mg, 0.12 mmol, 1.4 eq) and N,N-diisopropylethylamine (93 μL, 0.53 mmol, 6 eq) in DMF (0.4 mL). After stirring at room temperature for 1 h the reaction mixture was precipitated in diethyl ether (60 mL). Centrifugation (5 min at 4000 rpm) was followed by decantation and the solid was washed with ether (10 mL). The centrifugation procedure was repeated and the resulting solid was dried in vacuo for 1 h. The compound was purified with preparative RP-HPLC using a gradient of 33 to 36% MeCN in H₂O (both containing 0.1 v/v % formic acid). Lyophilization yielded pure 3 (106 mg, 61 μmol, 70%) as a white fluffy solid. ESI-MS: m/z Calc. for C₈₀H₁₃₈N₁₆O₂₅ 1723.00; Obs. [M+3H]³⁺ 575.42, [M+2H]²⁺ 862.67, [M+H]⁺ 1723.42.

### Synthesis of UPy-OEG-G (4): 42-Amino-39,42-dioxo-3,6,9,12,15,18,21,24,27,30,33,36-dodecaoxa-40-azadotetracontyl (12-(3-(6-(3-(6-methyl-4-oxo-1,4-dihydropyrimidin-2-yl)ureido)hexyl)ureido)dodecyl)carbamate

A solution of 2 (618 mg, 0.48 mmol) in CHCl₃/DMF 1:2 (5 mL) was added to a stirring solution of glycinamide hydrochloride (66 mg, 0.59 mmol, 1.2 eq) and N,N-diisopropylethylamine (0.50 mL, 2.8 mmol, 6 eq) in DMF (0.5 mL). After stirring at room temperature for 1 h the solvent was removed in vacuo (oil pump, 45° C.). The resulting solid was dissolved in CHCl₃/MeOH 9:1 (250 mL) and washed with H₂O/brine 1:1 (2×100 mL). After drying with Na₂SO₄ and filtration, the filtrate was evaporated to dryness and the resulting solid was flushed with CHCl₃ (2×20 mL). The solid was re-dissolved in CHCl₃/MeOH 3:1 (4 mL) and precipitated in diethyl ether (35 mL). Centrifugation (5 min at 4000 rpm) was followed by decantation and the solid was washed with ether (20 mL). Centrifugation and decantation were repeated and the solid was re-dissolved in CHCl₃/MeOH 3:1 (4 mL). The entire precipitation-centrifugation procedure was repeated once and the resulting solid was dried in vacuo for 16 h, yielding pure 4 (534 mg, 0.45 mmol, 93%) as a white solid. ESI-MS: m/z Calc. for C₅₅H₁₀₃N₉O₁₉ 1193.74; Obs. [M+2H]²⁺ 597.92, [M+H]+1194.58, [M+Na]⁺ 1216.58.

### Synthesis of UPy-OEG-Cy5 (5)

Reverse-phase high-performance liquid chromatography-mass spectrometry (RP-HPLC-MS) was performed on a Thermo scientific LCQ fleet spectrometer. Sulfo-Cy5-NH₂ was purchased from Lumiprobe (USA).

The route of synthesis of the UPy-OEG-Cy5 (5) is depicted below. Starting material was synthesized as described by Hu, et al. Long-Term Expansion of Functional Mouse and Human Hepatocytes as 3D Organoids (Cell 2018, 175 (6): 1591-1606.e1519).

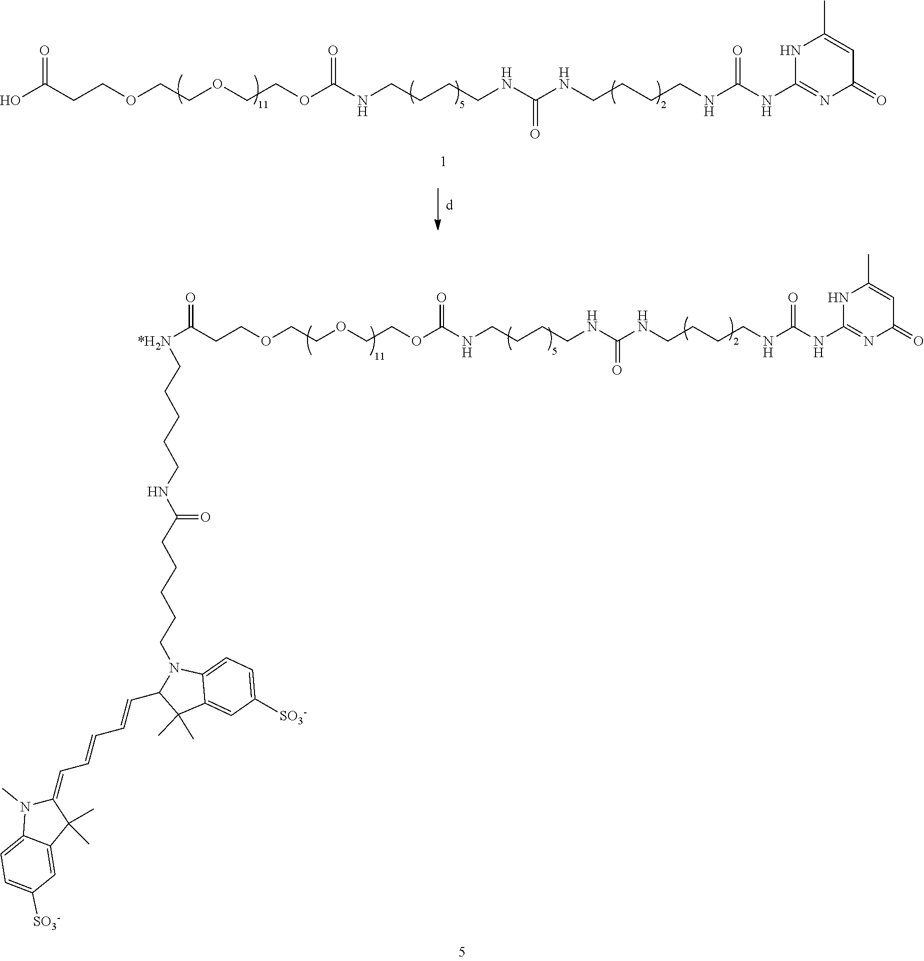

where:
(d) is 1-[Bis(dimethylamino)methylene]-1H-1,2,3-triazolo[4,5-b]pyridinium 3-oxid hexafluorophosphate, Hexafluorophosphate Azabenzotriazole Tetramethyl Uronium, N-N-diisopropylethylamine, Sulo-Cy5-NH2, DMF, r.t., 1 h, 45%.

### Synthesis of 1-[6-[(6-aminohexyl)amino]-6-oxohexyl]-2-[5-(1,3-dihydro-1,3,3-trimethyl-5-sulfo-2H-indol-2-ylidene)-1,3-pentadien-1-yl]-3,3-dimethyl-5-sulfo-1-((6-methyl-4-oxo-1,4-dihydropyrimidin-2-yl)amino)-1,10,25-trioxo-26,29,32,35,38,41,44,47,50,53,56,59,62-tridecaoxa-2,9,11,24-tetraazapentahexacontan-65-oate (5)

1-((6-Methyl-4-oxo-1,4-dihydropyrimidin-2-yl)amino)-1,10,25-trioxo-26,29,32,35,38,41,44,47,50,53,56,59,62-tridecaoxa-2,9,11,24-tetraazapentahexacontan-65-oic acid (1) (2.36 mg, 20.8 μmol), 1-[Bis(dimethylamino)methylene]-1H-1,2,3-triazolo[4,5-b]pyridinium 3-oxid hexafluorophosphate, Hexafluorophosphate Azabenzotriazole Tetramethyl Uronium (1.58 mg, 41.6 μmol) was dissolved in DMF (2 mL). N,N-Diisopropylethylamine (2.15 mg, 16.6 μmol) was added and the solution was stirred at room temperature for 15 min. Sulfo-Cy5-NH₂ (2 mg, 27.0 μmol) dissolved in DMF (3 mL) was added to the solution and stirred for 1 h at an argon environment. H₂O (containing 0.1 v/v% formic acid, 20 mL) was added to the solution and centrifuged (4 min, 3000 rpm) followed by decantation. Ultrapure water was added (20 mL) and the product was lyophilized. The compound (5) was purified with preparative RP-HPLC using a gradient of 40% ACN in H₂O (both containing 0.1 v/v% formic acid). Lyophilization yielded pure 3 (1.75 mg, 9.4 μmol, 45%) blue solid. ESI-MS: m/z Calc. for C₉₁H₁₄₉N₁₁O₂₅S₂ 1861.37; Obs. [M+3H]³⁺ 621.33, [M+2H]²⁺ 931.17, [M+H]+1861.75.

### In Vitro Degradation and Release Behavior

Hydrogels (100 μL), containing 100 μM of UPy-OEG-Cy5 or Cy5, were formed at the bottom of 2 mL glass vials through the pH-induced gelation. 500 μL of PBS was added into each vial, and the vials were incubated at 37° C. for up to 7 days. At each time point, the solution was collected using a pipette, the extra water at the surface of hydrogels was removed using tissue wipers, the weight of the hydrogels was recorded using a microbalance to determine their weight change, and 500 μL of fresh PBS was added into each vial. At the last time point, the hydrogels were dried in a vacuum oven at 60° C. overnight, after which their dry mass was measured using a microbalance to determine their erosion. The concentration of released UPy-OEG-Cy5 or Cy5 molecules in solutions was determined by measuring the fluorescence intensity using a Tecan Safire² microplate reader (646 nm excitation, 662 nm emission). The amount of released dye (UPy-OEG-Cy5 or Cy5) was calculated as percentage of the initial dye content incorporated in the hydrogels.

### Organoid Formation and Culture, Human Hepatocyte Organoid Culture

Human hepatocyte organoids isolated from human fetal tissue were cultured on Matrigel based on the culturing method as described by Hu, et al. Long-Term Expansion of Functional Mouse and Human Hepatocytes as 3D Organoids (Cell 2018, 175 (6): 1591-1606.e1519). Briefly, 20,000-50,000 cells were suspended in Matrigel, and 10 droplets of 10 μl were plated in a 6 wells plate. After Matrigel was solidified (>20 min), 1 ml Hepatocyte-medium was added per well. Human Hepatocyte-Medium consists of AdDMEM/F12 (Thermo Scientific, with HEPES, GlutaMax and Penicillin-Streptomycin) plus 15% RSPO1 conditioned medium (home-made), B27 (minus vitamin A), 50 ng/ml EGF (Peprotech), 1.25 mM N-acetylcysteine (Sigma), 10 nM gastrin (Sigma), 3 mm CHIR99021 (Sigma), 50 ng/ml HGF (Peprotech), 100 ng/ml FGF7 (Peprotech), 100 ng/ml FGF10 (Peprotech), 2 mM A83-01 (Tocris), 10 mM Nicotinamide (Sigma), 10 mM Rho Inhibitor g-27632 (Calbiochem) and 20 ng/ml TGFa. During culturing, the medium was refreshed every 2-3 days, and the organoids were usually passaged with a split ratio of 1:3 every 14 days.

### Organoid Encapsulation in Hydrogels

Pre-cultured human hypatocyte organoids in Matrigel were split in a 1:3 ratio and encapsulated in either UPy-based hydrogels or Matrigel. Organoids were resuspended in growth medium (60 μl) and mixed with a multifunctional, here a bifunctional UPy (bUPy) (120 μl, 0.75 wt %) in an Eppendorf tube.

The organoid encapsulation was carried out through the mixing-induced gelation method. To this end, the spheroids were included in monofunctional molecule dispersions. Thereafter, the monofunctional molecule dispersion (+organoid) was mixed with a multifunctional molecule dispersion inside an Eppendorf™ tube through gentle pipetting for ˜1 min. Thereafter, 50 μl of the mixture was pipetted in 3 droplets per well in a 12 wells plate. The Matrigel control was prepared separately and plated as 50 μl into 3 droplets in a 12 wells plate as well. All conditions were plated in triplicate. After 30 minutes of incubation at 37° C. and 5% CO₂ atmosphere, 1 ml of hepatocyte medium was slowly added to each well. The medium was refreshed at day 5, and brightfield images were recorded at day 1, day 5 and day 7. An ATP assay was performed on day 7 using CellTiter-Glo® 3D Cell Viability Assay. The data was corrected for the amount of organoids present. The change in surface area was determined by measuring the area of 30 organoids in ImageJ (FIG. 18) on both d1 and d7 of the same droplet in the same well, and calculating the fold change (FC) in surface area by the following equation: FC=([Area_{d7}]-[Area_{d1}])/[Area_{d1}].

### Cryogenic Transmission Electron Microscopy

Stock dispersions (10 mg/mL) of supramolecular fiber assemblies were prepared by dissolving multifunctional- and monofunctional-type molecules at different ratios in alkaline PBS solutions (containing 80 mM of NaOH), followed by the addition of HCl (final concentration=83 mM) for pH neutralization. Thereafter, Cryo-TEM sample preparation was done using dispersions with concentrations ranging from 0.05 to 10 mg/mL for optimal fiber visibility. Lacey carbon film grids (200 mesh, 50 μm hole size; Electron Microscopy Sciences) were surface plasma treated at 5 mA for 40 s using a Cressington 208 carbon coater, and 3 μL of each dispersion was applied into each grid hole. Using an automated vitrification robot (FEI Vitrobot™ Mark III), excess sample was removed by blotting using filter paper for 3 s at −3 mm. Thin films of dispersions were vitrified by plunging the grids into liquid ethane just above its freezing point. Imaging was carried out on a FEI-Titan TEM equipped with a field emission gun operating at 300 kV. Samples were imaged using a post-column Gatan energy filter and a 2048×2048 Gatan CCD camera. Micrographs were recorded at low dose conditions, using a defocus setting of 10 μm at 25000 magnification, or defocus setting of 40 μm at 6500 magnification. Contrast and brightness of images were manually adjusted using the ImageJ software to improve the visibility of fibers.

### Preparation of Hydrogels

pH-induced gelation: multifunctional- and monofunctional-type molecules were dissolved at 70° C. in an alkaline PBS solution. The PBS solution contained 80 mM or 160 mM NaOH for the preparation of hydrogels with wt % or 10 wt % solid contents, respectively. Thereafter, to initiate gelation, a specific volume of 1M HCl solution was added to the alkaline solution of building blocks and additives to reach neutral pH. The resulting mixture contained 83 mM or 113 mM of HCl for the hydrogels with wt % or 10 wt % polymer contents, respectively. The hydrogels were kept overnight in a 4° C. fridge to assure complete gelation.

Mixing-induced gelation: multifunctional- and monofunctional-type molecules were dissolved separately at 70° C. in alkaline PBS solutions containing 80 mM NaOH. Thereafter, a specific volume of 1M HCl solution was added to each solution at room temperature to reach neutral pH (final HCl concentration=83 mM). To initiate gelation, the resulting multifunctional and monofunctional molecule dispersions were mixed via pipetting.

### Rheological Characterizations

A discovery hybrid rheometer (DHR-3, TA Instruments) was used for rheological characterizations of supramolecular solutions and hydrogels. Hydrogel disks were made via the pH-induced gelation method inside cylindrical Teflon molds (diameter=8 mm, height=2 mm). Pre-formed hydrogel disks were analysed using a flat stainless-steel geometry (diameter=8 mm) at a gap height of 0.5-2 mm. Low viscosity silicon oil (47 V 100, RHODORSIL®) was applied to seal the gap around the hydrogel disks to minimize drying during the measurements at 37° C. Non-gelling samples were tested at 20° C. or 37° C. using flat stainless-steel (diameter=8 mm) or 2.007° cone-plate aluminium (diameter=20 mm, with solvent trap to minimize sample drying) geometries at gap heights of 500 μm or 56 μm, respectively. Mixing-induced gelation was evaluated using the cone-plate geometry at a gap height of 56 μm by mixing the dispersions on the Peltier plate using a pipette immediately prior to the measurements. Strain sweep measurements (1-1000% strain, 10 rad/s) were performed to determine the linear viscoelastic region of hydrogels. Frequency sweeps were carried out with frequencies ranging from 100 to 0.01 rad/s, at a constant strain of 1%. Time sweeps were carried out at a constant frequency and a constant strain of 10 rad/s and 1%, respectively. Stress relaxation experiments were performed by applying a strain of 1%, and monitoring the generated stress for 10 min. The data were normalized using the stress detected at 1 s for each sample.

### Fluorescence Recovery After Photo-Bleaching

FRAP measurements were carried out using a Leica TCS SP5 inverted confocal microscope (Leica Microsystems) equipped with a 20× objective (HCX PL APO CS 20.0×0.70 DRY UV). Hydrogels were formed through pH-induced gelation inside the cylindrical chamber (diameter=7 mm) of 35-mm dishes with cover glass bottoms (MatTek, Ashland, Mass.). Hydrogels contained 20 μM of UPy-OEG-Cy5 or 0.5 mg/mL of FITC-Dextran (Fluorescein isothiocyanate-dextran (average MW 100 kDa, or 2000 kDa; Sigma-Aldrich) for exchange dynamics and pore size measurements, respectively. To minimize sample drying during the measurements, the chamber was covered with a cover glass and sealed with nail polish, wet tissue paper was placed in the dish, and the lid was sealed with Parafilm®. Prior to each measurement, the sample was placed inside the environmental chamber of the microscope at 37° C. to equilibrate for 1 h. Exchange dynamics experiments were carried out via sample illumination using white laser at 646 nm wavelength for Cy5 excitation. Emission was collected at 660-700 nm wavelength using a hybrid detector. A circular area with a diameter of 20 pm was photo-bleached at 60% laser power for 31 frames (0.653 frame/s), and the post-bleaching time-lapse imaging was performed for >12 h. Data normalization was conducted via dividing the fluorescence intensity in the bleached area by the fluorescence intensity in a non-bleached circular area of same size in each image. T_{1/2} and mobile fraction were determined using the easyFRAP software as described by Rapsomaniki, et al. easyFRAP: an interactive, easy-to-use tool for qualitative and quantitative analysis of FRAP data (Bioinformatics 2012, 28 (13): 1800-1801) by means of a double exponential fitting. The initial rate of recovery was determined by calculating the slope of the linear regression fit of the recovery curve for the first 60 s of post-bleaching.

Experiments concerning pore size evaluation were carried out via sample illumination using white laser at 493 nm wavelength for FITC excitation. Emission was collected at 520 nm wavelength using a hybrid detector. A circular area with a diameter of 20 μm was photo-bleached at 60% laser power for 15 frames (0.653 frame/s), and the post-bleaching time-lapse imaging was performed for 5 min. Data normalization was performed as described above, and the initial rate of recovery was calculated for the first 2 s of post-bleaching.

### Cell Culture

Human vena saphena cells (HVSCs) were harvested from the human vena saphena magna, following the Dutch guidelines for secondary use of materials. HVSC expansion and culture was performed in Dulbecco's modified Eagle's medium (DMEM; Gibco) supplemented with 10% fetal bovine serum (FBS; Greiner Bio one), 1% GlutaMax™ (Gibco) and 1% penicillin/streptomycin (Lonza).

L9TB cardiomyocyte progenitor cells (CMPCs) were immortalized through lentiviral transduction of hTert and BMI-1. CMPC expansion and culture was performed in SP++ growth medium, composing 3:1 volumetric mixture of M199 (Gibco) and EGM-2 BulletKit (Lonza), supplemented with 10% FBS (Greiner bio-one), 1% non-essential amino acids (Gibco), and 1% penicillin/streptomycin (Lonza). CMPC expansion was carried out in gelatin-coated flasks. For all the experiments, the culture medium was refreshed every three days.

2D cell culture experiments were carried out by forming the hydrogels (70 μL) through pH-induced gelation inside 96-well cell culture plates. Prior to cell seeding, the hydrogels were incubated with ˜200 μL of culture medium for 15 min to ensure physiological pH and ionic concentration during the cell culture. Thereafter, the medium was removed, 200 μL of cell suspension containing 250,000 cells (1.25 million cells/mL) were added into each well, and the plates were incubated (37° C., 5% CO₂) for 1 or 3 days. At each time points, the medium was removed and the samples were washed with PBS to remove nonadherent cells.

3D cell culture experiments were carried out by encapsulation of cells in hydrogels (0.5×10\hat{ }6 cells/mL) using the mixing-induced gelation method. To this end, cells were included in the supramolecular dispersion containing monofunctional molecules (monofunctional molecule dispersion). Thereafter, 50 μL of the monofunctional molecule dispersion were mixed with 50 μL of the multifunctional molecule dispersion (+cells) inside each well of 8-well chambered cover glasses using a pipette. The resulting mixtures were kept in an incubator for 15 min for completion of the gelation step. Thereafter, ˜200 μL of culture medium was added into each well and the cells were cultured for up to 7 days. To block exocytosis, 120 nM of Exo-1 (Sigma Aldrich) was added to culture media and replenished every day. To inhibit local MMP activity, 5 nM of recombinant TIMP-3 (R&D Systems) was encapsulated in the hydrogels, added to culture media, and replenished daily.

### Quantification of Number of Adhered Cells

The number of cells adhered to the surface of hydrogels was quantified using a CyQuant® Cell Proliferation Assay (Invitrogen), following the manufacturer's guideline. The assay measures the DNA content in cell lysis by utilizing a dye that displays strong fluorescence enhancement upon binding to nucleic acids. A standard curve was plotted using known cell concentrations, which was used to translate the fluorescence intensity to cell number for each sample.

### Cell Staining and Imaging

Actin cytoskeleton and nuclei staining were performed using Phalloidin-FITC and 4′,6-diamidino-2-phenylindole (DAPI), respectively. Prior to the staining, the cells were fixated with 3.7% formaldehyde, washed twice with PBS, and permeabilized with 0.5% Triton X-100. Live/Dead staining was carried out according to the manufacturer's manual (Thermo Fisher Scientific) using calcein-AM and propidium iodide to stain for live and dead cells, respectively. A Leica TCS SP5 inverted confocal microscope (Leica Microsystems) was used to acquire z-stack images using 10× (HCX PL APO CS 10.0×0.40 DRY UV) and 63× (HCX PL APO CS 63.0×1.20 WATER UV) objectives.

### Cell Morphology and Viability Analyses

Cell morphology was analysed from maximum-intensity z-projections of images obtained after actin cytoskeleton and nuclei staining. To this end, the ImageJ software was used to determine the circularity and the length of the longest axis of individual cells. The cell circularity was calculated as 47 times the cell area, divided by the square of cell perimeter. Cell viability was calculated by counting live (green) and dead (red) cells in maximum-intensity z-projections of microscopy images.

### Spheroid Formation and Culture

Spheroid formation: Cell suspensions were prepared at a concentration of 25000 cells/mL. 200 μL of cell suspension was added into each well of non-adhesive round bottom 96-well plates (Nunclon™ Sphera™, Thermo Fisher). The plates were centrifuged for 2 min at 200 RCF, and incubated (37° C., 5% CO2) for 5 days for spheroid formation. Thereafter, the spheroids were collected into Eppendorf™ tubes using a pipette. The spheroid density in medium was adjusted to 360 spheroids/mL through centrifugation for 2 min at 300 RCF.

Spheroid encapsulation: The spheroid encapsulation was carried out through the mixing-induced gelation method. To this end, the spheroids were included in monofunctional molecule dispersions. Thereafter, the monofunctional molecule dispersion was mixed with a multifunctional molecule dispersion (+spheroids) inside an Eppendorf™ tube through gentle pipetting for ˜30 s. Thereafter, the mixtures (100 μL) were pipetted onto 8-well chambered cover glasses, and placed in an incubator (37° C., 5% CO₂) for 20 min for completion of the gelation step. Thereafter, ˜200 μL of culture medium was added into each well, and the spheroids were cultured for up to 14 days. The culture medium was refreshed every three days.

Spheroid imaging, staining and extraction: A phase contrast microscope (Invitrogen™ EVOS™ XL Digital Inverted Microscope) was used to image the spheroids at different time points. At Day 14, Live/Dead staining was carried out according to the manufacturer's manual (Thermo Fisher Scientific) using calcein-AM and propidium iodide, and the spheroids were imaged using a Leica TCS SP5 inverted confocal microscope (Leica Microsystems). Thereafter, the spheroids were extracted from the hydrogels through gentle mechanical disruption of the gel networks using pipette tips. The extracted spheroids were seeded onto 8-well chambered cover glasses, and cultured (37° C., 5% CO₂) for 2 days with ˜200 μL of culture medium. Thereafter, Live/Dead imaging was carried out using a Leica TCS SP5 inverted confocal microscope (Leica Microsystems).

Quantification of cell migration distance: The cell migration was quantified by measuring the average distance that the cells move from the initial surface of spheroids (at Day 0 of encapsulation) toward the surrounding hydrogel matrix. To this end, phase contrast microscopy images of spheroids were analysed using the ImageJ software. Accordingly, the longest distance between any two points (Feret's diameter; D_{F}) of the objects composed of the spheroids and their migrating cells was quantified. The cell migration distance from individual spheroids at each time point was calculated as D_{F} minus D_{F(Day 0)}, divided by two.

### Sample ID Used

Table 1 provides an overview of the different dispersions used throughout the examples. It is noted that the sample ID identifies the specific dispersion comprising multifunctional hydrogelators (in this case bifunctional hydrogelators) and monofunctional hydrogelators by their weight percentages based on the total weight of the dispersion. Table 1 also includes the molar ratio of the dispersions used.

TABLE 1
overview of sample IDs as used in the drawings and molar ratios of
components present in the hydrogel formulations tested.

| | | | Molar ratio |
|---|---|---|---|
| Sample ID | Multifunctional (B) | Monofunctional (M) | (B/M) |
| B5 | 5.0 wt % | 0.0 wt % | — |
| B4.5M0.5 | 4.5 wt % | 0.5 wt % | 1/1 |
| B3.5M1.5 | 3.5 wt % | 1.5 wt % | 1/4 |
| B2.5M2.5 | 2.5 wt % | 2.5 wt % | 1/9 |
| B1.5M3.5 | 1.5 wt % | 3.5 wt % | 1/22(21*) |
| B0.5M4.5 | 0.5 wt % | 4.5 wt % | 1/84(81*) |
| M5 | 0.0 wt % | 5.0 wt % | — |
| B0.25M2.25 | 0.25 wt % | 2.25 wt % | 1/84 |
| B1M9 | 1.0 wt % | 9.0 wt % | 1/84 |
| B0.125M1.125 | 0.125 wt % | 1.125 wt % | 1/84 |
| B0.063M0.563 | 0.063 wt % | 0.563 wt % | 1/84 |
| B0.031M0.281 | 0.031 wt % | 0.281 wt % | 1/84 |
| B0.002M0.018 | 0.002 wt % | 0.018 wt % | 1/84 |

*For hydrogels containing 3 mM of UPy-OEG-cRGD, due to the difference between molecular weights of UPy-OEG-G and UPy-OEG-cRGD.

### DESCRIPTION OF THE DRAWINGS

FIG. 1. Supramolecular building blocks and their self-assembly process

FIG. 1A. Molecular structures of the supramolecular building blocks and additives.

FIG. 1B. Schematic illustration of the self-assembly process of multifunctional (B) and monofunctional (M) molecules into fibers. Ureido-pyrimidinone (UPy)-based molecules self-assemble into fibers at physiological pH and temperature, through dimerization via quadruple hydrogen bonds (grey units), and lateral stacking based on urea moieties (red units).

FIG. 1C. Representative Cryo-TEM images showing the morphology of fibers assembled from M and B molecules at different molar ratios (M/B). Scale bars, 100 nm.

FIG. 2. Hydrogels assembled from different ratios of supramolecular building blocks

FIG. 2A. Digital photographs showing different supramolecular compositions subjected to the inverted-vial test.

FIG. 2B. Frequency dependence of storage (G′) and loss (G″) moduli of different compositions of supramolecular hydrogels.

FIG. 2C. G′ and damping factor (tan(delta)) values of hydrogels measured at 1 rad/s and 1% strain.

FIG. 2D. Stress relaxation behaviour of supramolecular hydrogels measured by subjecting the hydrogels to 1% strain.

FIG. 2E. Fluorescence recovery after photo-bleaching (FRAP) tests performed on hydrogels containing 20 μM of UPy-OEG-Cy5 supramolecular additives. Quantified results show the timespan (τ½) during which the Cy5 fluorescence intensity recovers to half its original value, the fraction of fluorescence intensity that recovers when fluorescence intensity curves reach plateau values (Mobile fraction (%)), and the rate of fluorescence recovery during the first 60 s after photo-bleaching (Initial rate (s-1)).

FIG. 2A-2E. All hydrogels contain a total polymer content of 5 wt %. All data are shown as mean±s.d.

FIG. 3. Cell adhesion and spreading on hydrogel with different compositions

FIG. 3A. Representative images of HVSCs after 1 day of culture on different supramolecular hydrogel compositions.

FIG. 3B. Number of cells adhered onto hydrogel surfaces after 1 and 3 days of culture. PS indicates polystyrene control.

FIG. 3C. Length of longest axis and d, circularity of cells after 1 day of culture on supramolecular hydrogels with different compositions.

FIG. 3A-3D. Hydrogels contain 3 mM of UPy-OEG-cRGD supramolecular additives.

FIG. 3E, 3F. Representative images of HVSCs after 1 day of culture and number of cells adhered after 3 days of culture on B0.5M4.5 supramolecular hydrogels containing different concentrations of UPy-OEG-cRGD supramolecular additives or 3 mM of cRGD.

FIG. 3A, 3E. Green and blue colours in images indicate actin and nucleus staining, respectively.

FIG. 3B-3D, 3F. NS, ** and **** indicate Not Significant, p<0.01, and p<0.0001, respectively. All data are shown as mean±s.d.

FIG. 4. Hydrogels assembled from different concentrations of supramolecular building blocks

FIG. 4A. Frequency dependence of viscoelastic behaviour, and quantified G′ and damping factor (tan(delta)) values (at 1 rad/s and 1% strain) of hydrogels with different polymer concentrations but a fixed M/B ratio.

FIG. 4B. Stress relaxation behaviour of supramolecular hydrogels measured by subjecting the hydrogels to 1% strain.

FIG. 4C. Fluorescence recovery after photo-bleaching (FRAP) tests performed on hydrogels containing 20 μM of UPy-OEG-Cy5 supramolecular additives. Quantified results show the timespan (τ½) during which the Cy5 fluorescence intensity recovers to half its original value, the fraction of fluorescence intensity that recovers when fluorescence intensity curves reach plateau values (Mobile fraction (%)), and the rate of fluorescence recovery during the first 60 s after photo-bleaching (Initial rate (s-1)).

FIG. 4D. Representative images of HVSCs after 1 day of culture on hydrogels with different polymer concentrations. Green color in images indicates actin staining.

FIG. 4E. Number of cells adhered onto hydrogel surfaces after 1 and 3 days of culture.

FIG. 4F, 4G. Length of longest axis and circularity of cells after 1 day of culture on supramolecular hydrogels with different polymer concentrations.

FIG. 4D-4G. Hydrogels contain 3 mM of UPy-OEG-cRGD supramolecular additives.

FIG. 4E-4G. NS, **, and *** indicate Not Significant, p<0.01, and p<0.001, respectively. All data are shown as mean±s.d.

FIG. 5. Cell encapsulation and spreading in supramolecular hydrogels

FIG. 5A. Viscoelastic properties of dispersions of 4.5 wt % M and 0.5 wt % B supramolecular fibers, and their mixture at physiological pH and temperature.

FIG. 5B. Schematic illustration of cell encapsulation in hydrogels via mixing of preassembled supramolecular fibers.

FIG. 5C. Representative images of HVSCs encapsulated within supramolecular hydrogels, after live (green color) and dead (red color) staining.

FIG. 5D. Quantification of viability of cells encapsulated in hydrogels.

FIG. 5E. Representative images of HVSCs encapsulated in supramolecular hydrogels of B0.25M2.25 composition without (−cRGD) or with (+cRGD) 3 mM of UPy-OEG-cRGD supramolecular additives after 3 days of culture. During the culture period, additional Exo-1 (120 nM) or TIMP-3 (5 nM) treatments are carried out to block exocytosis and protein remodelling, respectively. Green and blue colors in images indicate actin and nucleus staining, respectively.

FIG. 5F, 5G. Length of longest axis and circularity of cells after 3 day of culture in supramolecular hydrogels.

FIG. 6. Multicellular spheroids encapsulated in supramolecular hydrogels.

FIG. 6A. Representative images of HVSC and CMPC spheroids encapsulated in supramolecular hydrogels composition without (−cRGD) or with (+cRGD) 3 mM of UPy-OEG-cRGD supramolecular additives. Scale bars, 500 μm (main images) and 50 μm (insets).

FIG. 6B. Quantification of migration distance of cells from the initial surface of spheroids to hydrogel matrices. NS and ****, indicate Not Significant and p<0.0001, respectively. Data are shown as mean±s.d.

FIG. 6C. Representative images of HVSC and CMPC spheroids after 14 days of culture in supramolecular hydrogels. Green and red colors indicate live and dead cells, respectively. Scale bars, 200 μm. All hydrogels are of B0.25M2.25 composition.

FIG. 7. HVSC and CMPC spheroids

FIG. 7A, 7B. Extracted HVSC (A) and CMPC (B) spheroids after two days of culture on glass bottom culture plates. Green and red colors indicate live and dead cells, respectively. Scale bars, 500 μm.

FIG. 8. Cryo-TEM images

Overview cryo-TEM images showing the morphology of fibers assembled from M and B molecules at different molar ratios (M/B).

FIG. 9. Frequency dependence of storage (G′) and loss (G″) moduli

Frequency dependence of storage (G′) and loss (G″) moduli of different compositions of supramolecular samples with M5 composition. Data are shown for n=3 independent tests, and as mean±s.d.

FIG. 10. Viscoelastic behaviour of hydrogels

Frequency dependence of viscoelastic behaviour of hydrogels with different polymer concentrations but a fixed M/B ratio, measured at 1% strain.

FIG. 11. Viscoelastic and stress relaxation behaviour of hydrogels

FIG. 11A. Frequency dependence of viscoelastic behaviour of B0.5M4.5 hydrogels with or without UPy-OEG-cRGD additives, measured at 1% strain.

FIG. 11B. Stress relaxation behaviour of B0.5M4.5 hydrogels, with or without UPy-OEG-cRGD additives, measured by subjecting the hydrogels to 1% strain.

FIG. 12. Weight change, erosion and, additive release from hydrogels

FIG. 12A-12C. Weight change (A), erosion (B) and, UPy-OEG-Cy5 or Cy5 additive release (C) from the supramolecular hydrogels during an immersion test at 37° C. Hydrogels contained 100 μM of UPy-OEG-Cy5 or Cy5 additives. All values are presented as mean±s.d. for n=3 per experimental group.

FIG. 13. PEG content

Estimated PEG content of hydrogels with different compositions.

FIG. 14. CMPCs

Representative images showing CMPCs after 1 day of culture on M0.5B4.5 without or with UPy-OEG-cRGD additives. Green and blue colours in images indicate actin and nucleus staining, respectively.

FIG. 15. HVSCs

Representative image showing clusters of HVSCs after 1 day of culture on M1B9 hydrogels containing 3 mM of UPy-OEG-cRGD additives. Green colour in the image indicates actin staining.

FIG. 16. Longest axis length of HVSCs

Length of longest axis and circularity of HVSCs encapsulated in supramolecular hydrogels without or with 3 mM of UPy-OEG-cRGD additives, after 1 or 3 days of culture. *, p<0.05; ****, P≤0.0001; one-way analysis of variance (ANOVA) followed by Bonferroni post hoc. Results were obtained from 3 biologically independent experiments per group, and all values are shown as mean±s.d.

FIG. 17. FRAP test

FIG. 17A. Fluorescence recovery after photo-bleaching (FRAP) tests performed on supramolecular hydrogels containing 0.5 mg/mL of FITC-Dextran.

FIG. 17A-17D. Quantified FRAP results showing (B) the timespan (τ½) during which the fluorescence intensity recovers to half its original value, (C) the fraction of fluorescence intensity that recovers when fluorescence intensity curves reach plateau values (Mobile fraction (%)), and (D) the rate of fluorescence recovery during the first 2 s after photo-bleaching (Initial rate (s-1)).

FIG. 18. Organoid encapsulation and spreading in supramolecular hydrogels

FIG. 18A. Representative optical microscopy images showing the morphology of hepatic liver organoids encapsulated in different hydrogels, upon culture for 1 and 7 days.

FIG. 18B. Relative ATP level of the organoids upon 7 days of culture within different hydrogels. PS indicates the polystyrene control.

FIG. 18C. Fold change in surface area of the organoids from day 1 to day 7 of the culture period.

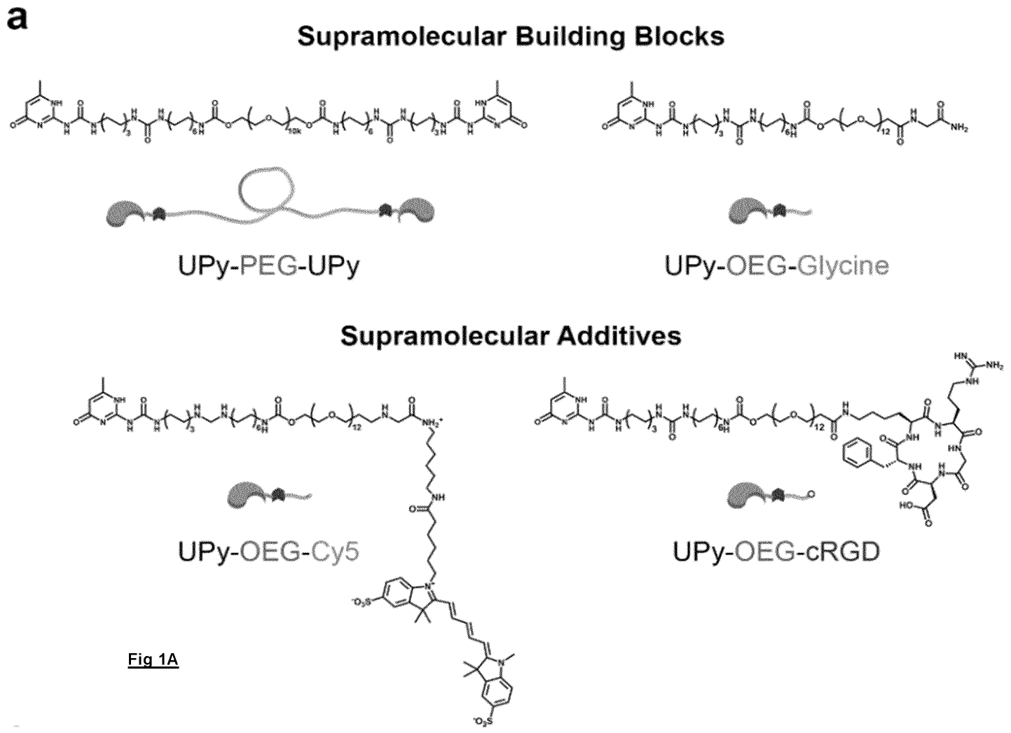

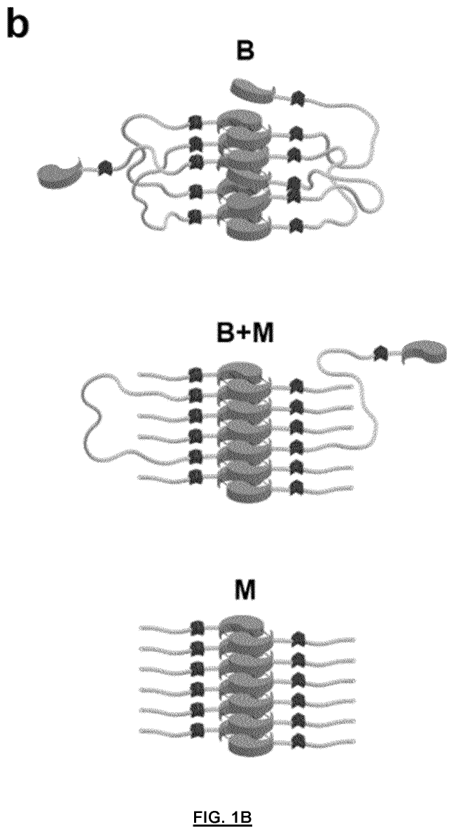

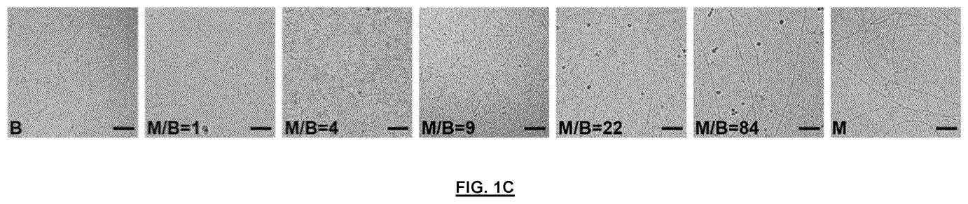

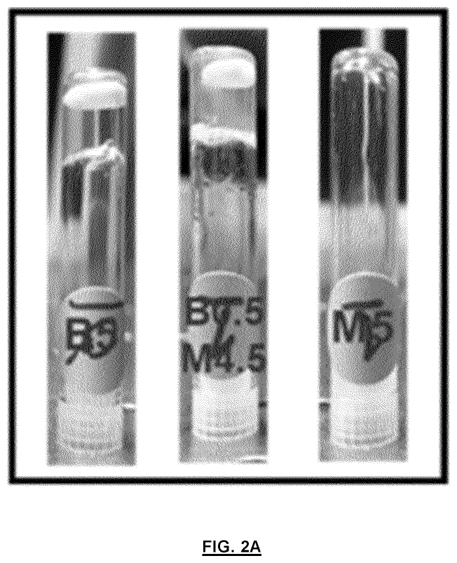

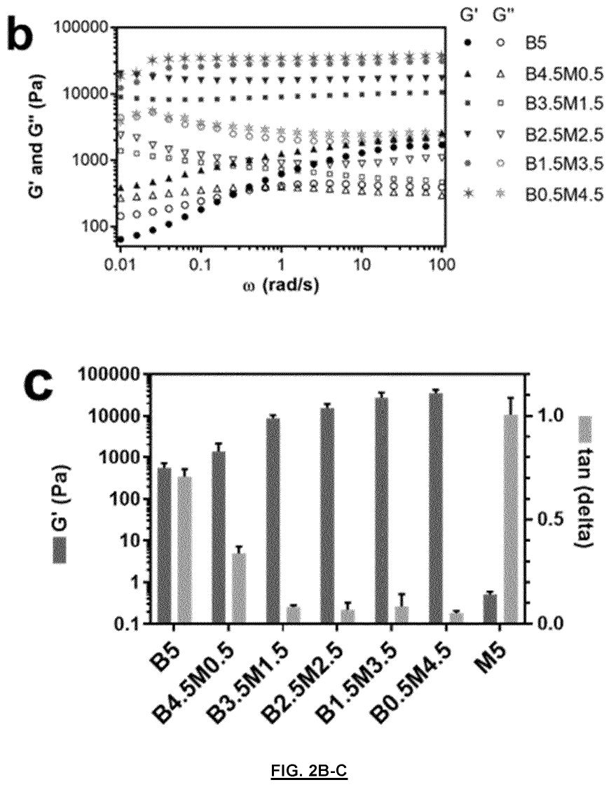

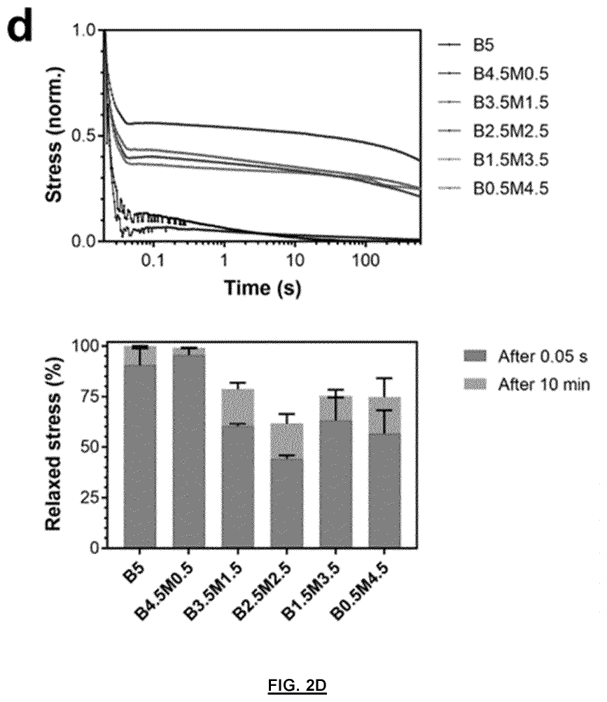

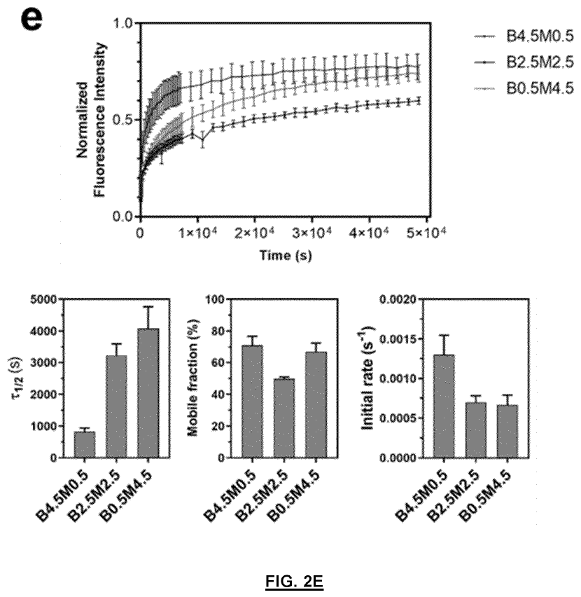

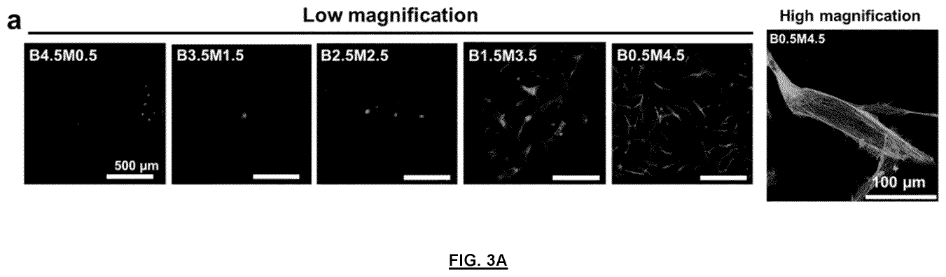

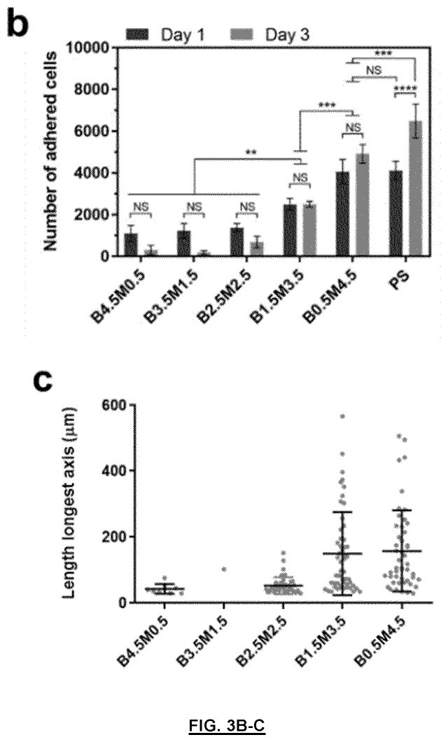

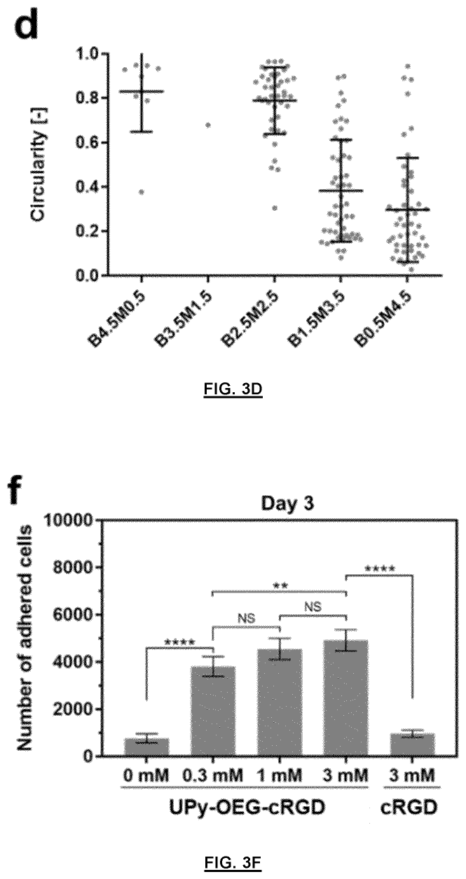

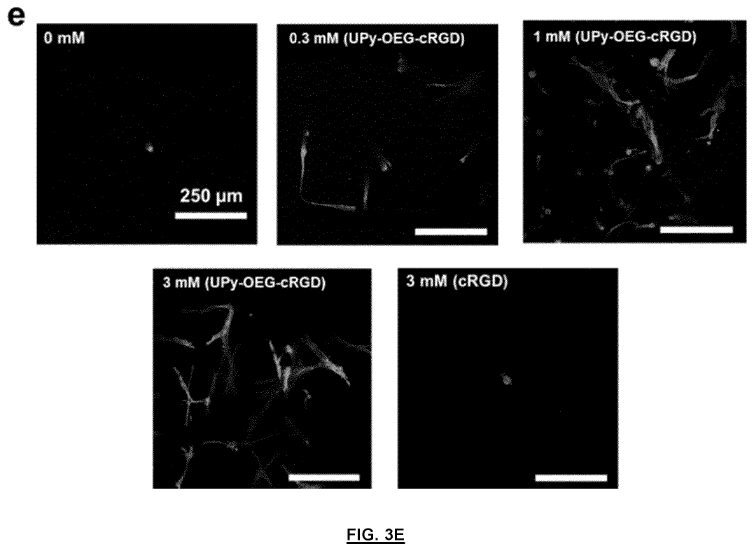

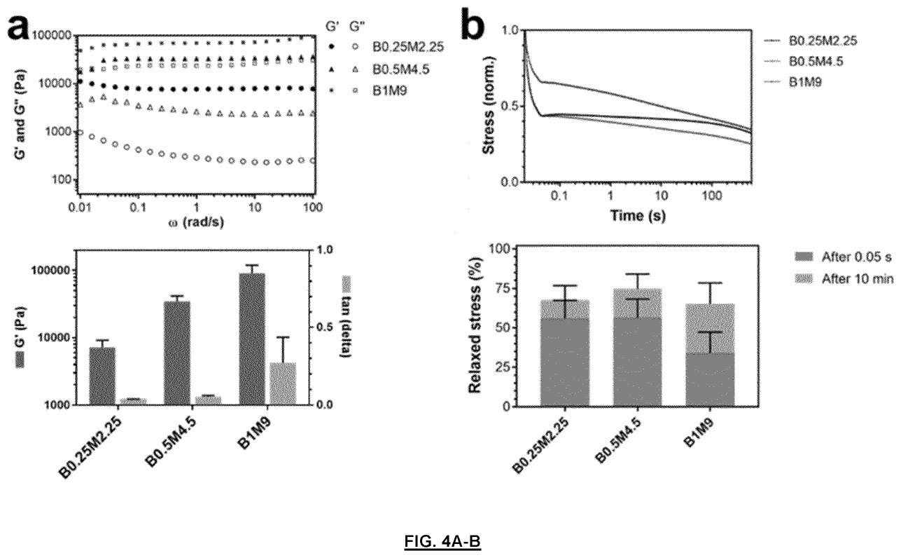

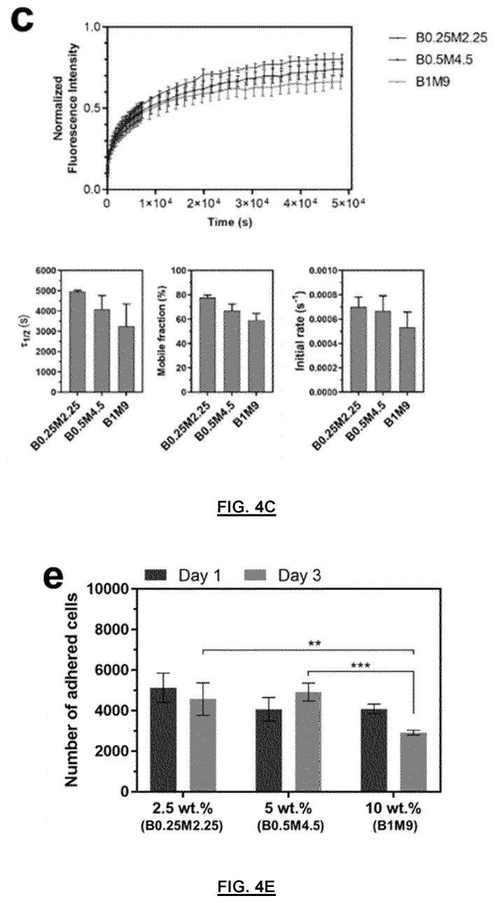

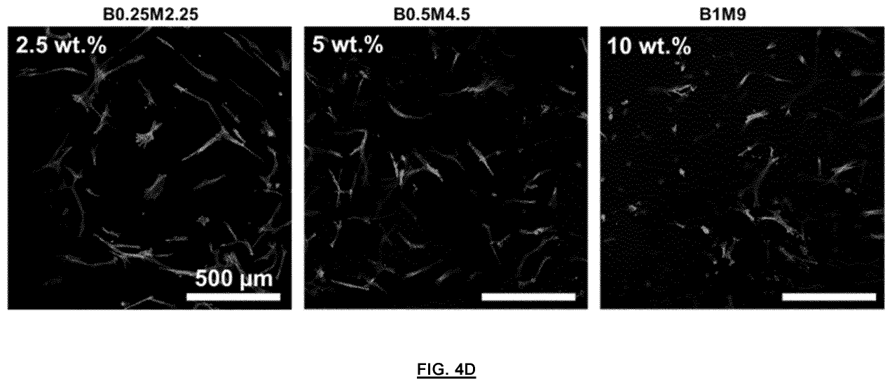

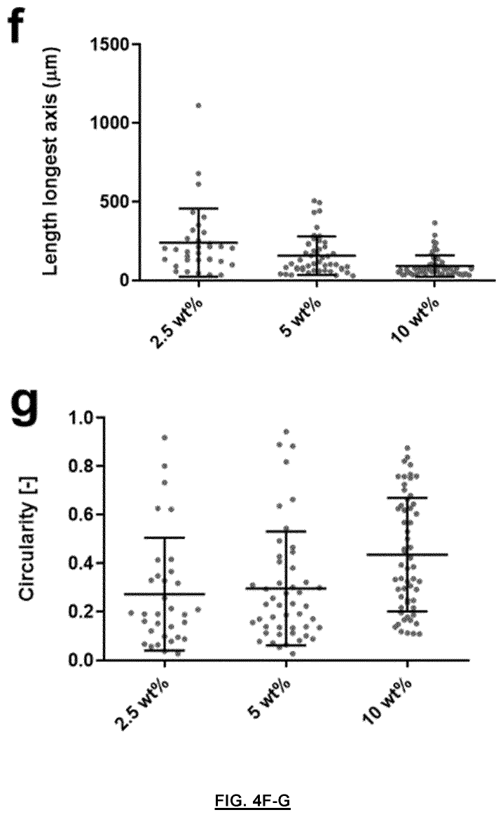

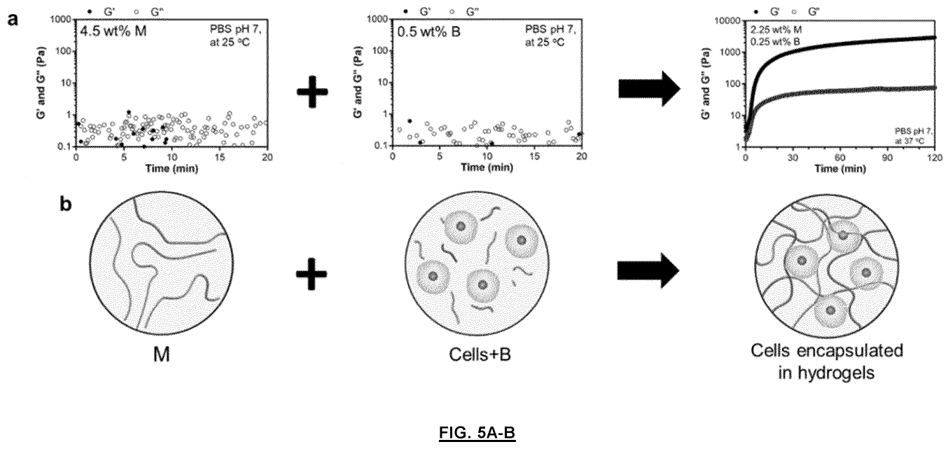

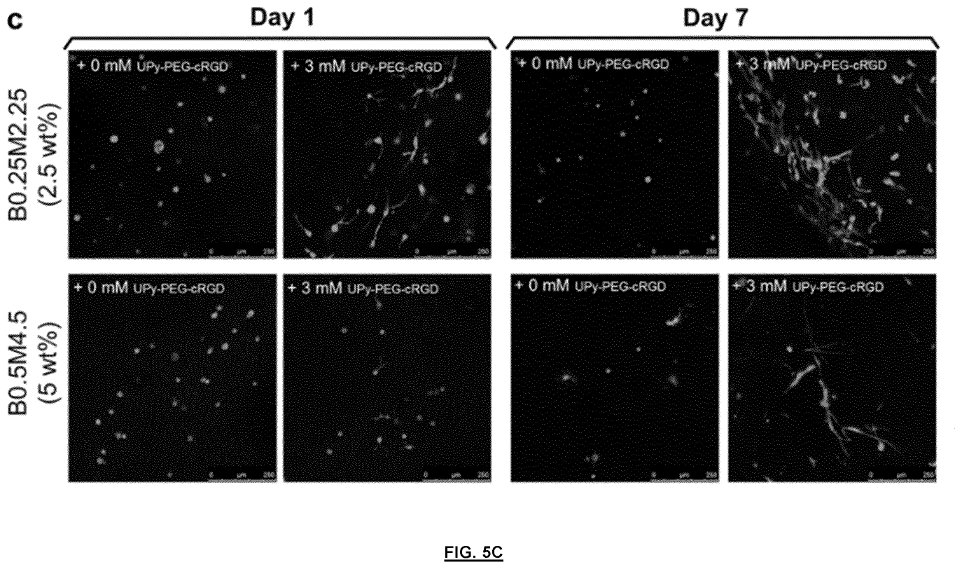

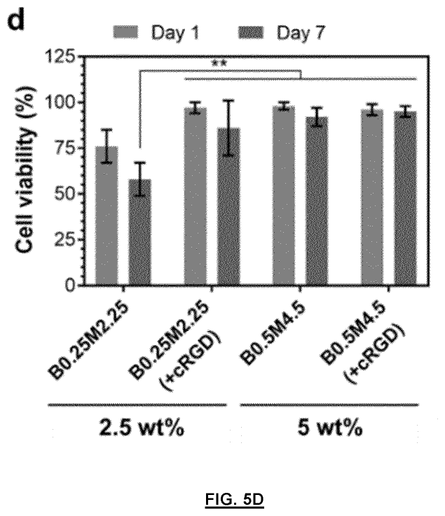

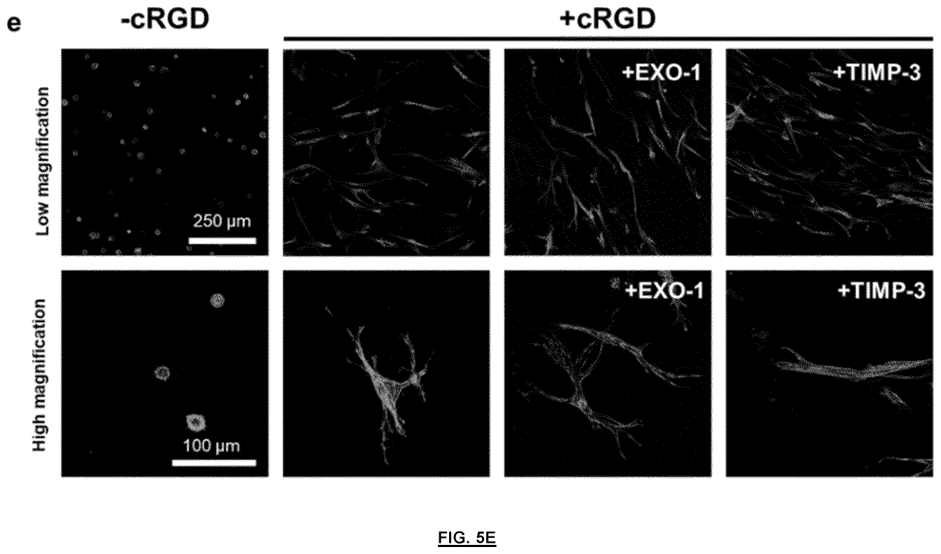

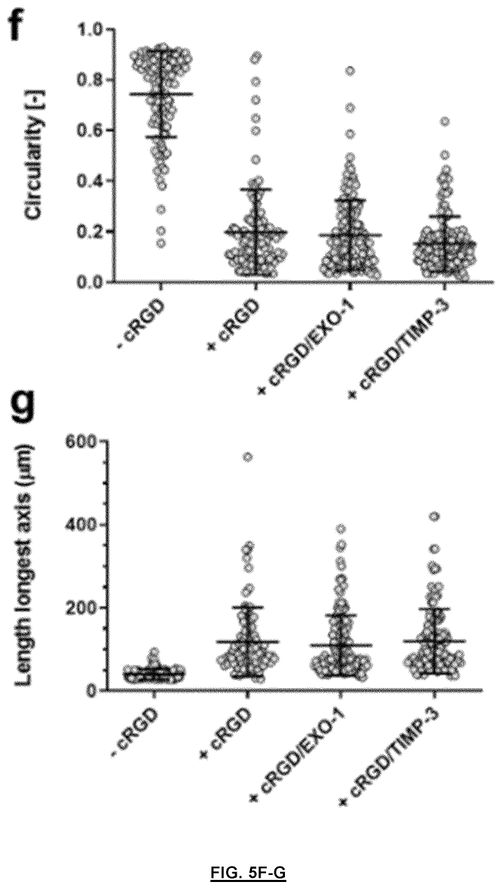

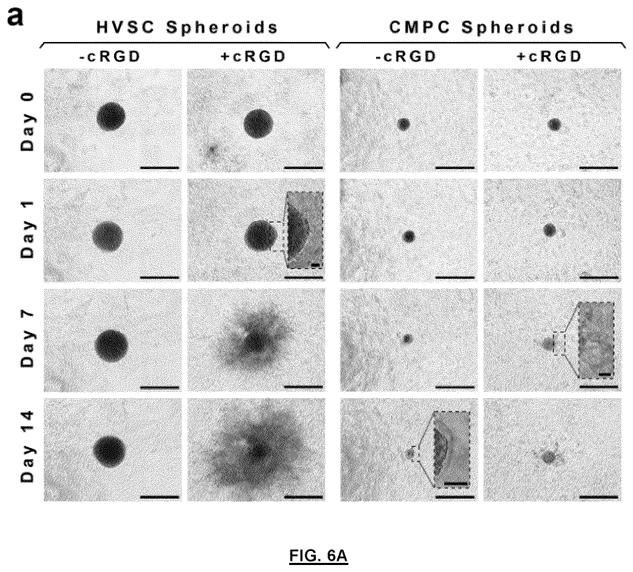

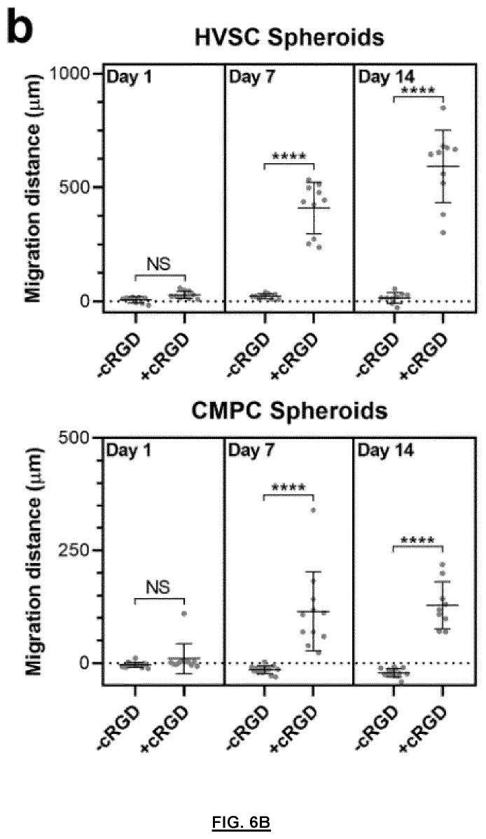

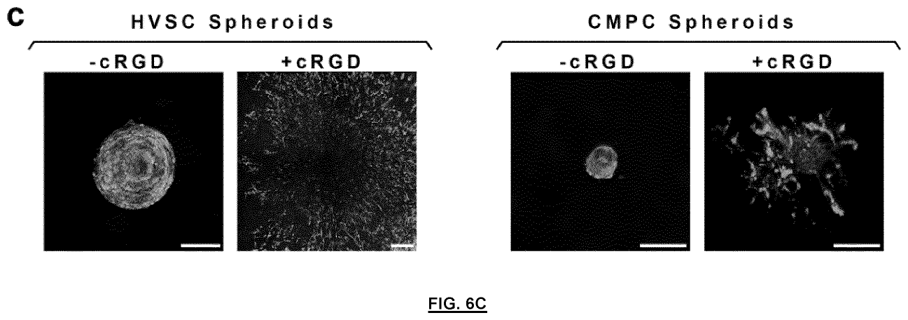

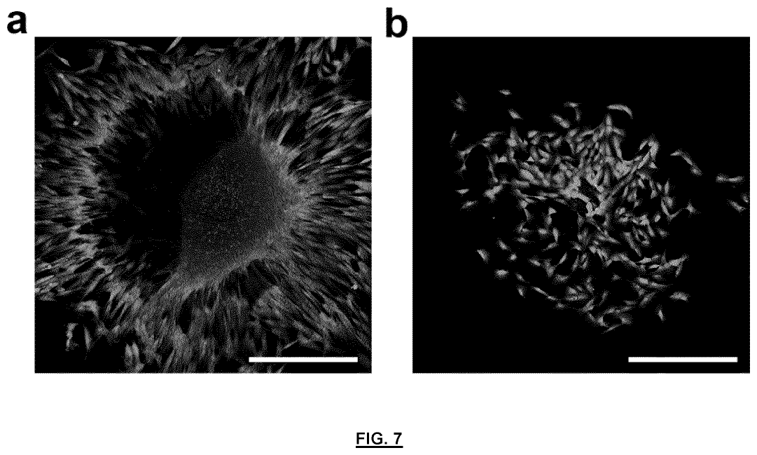

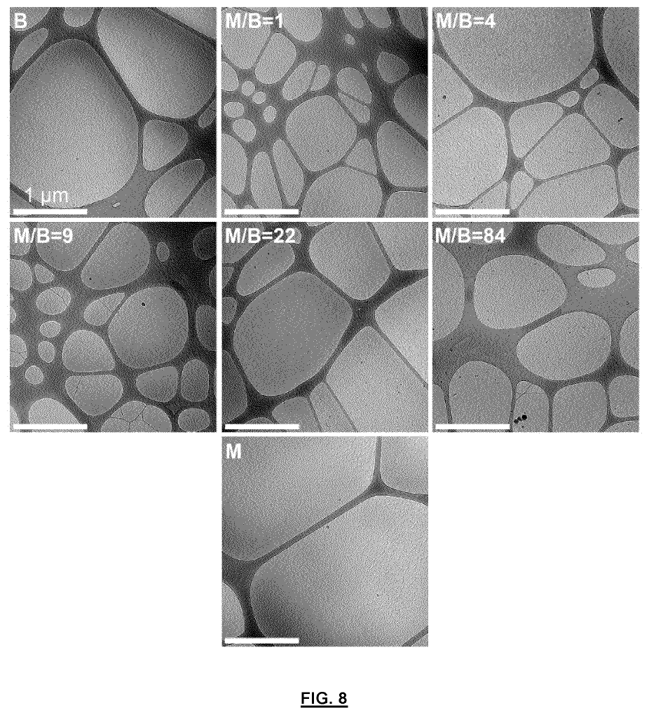

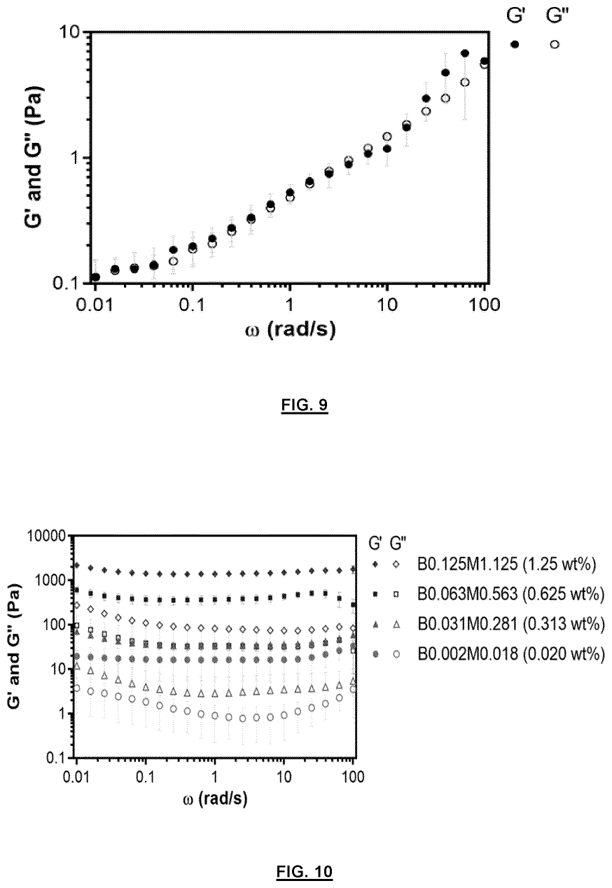

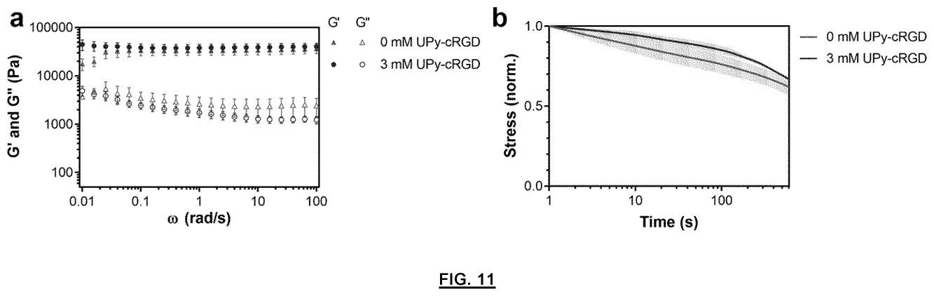

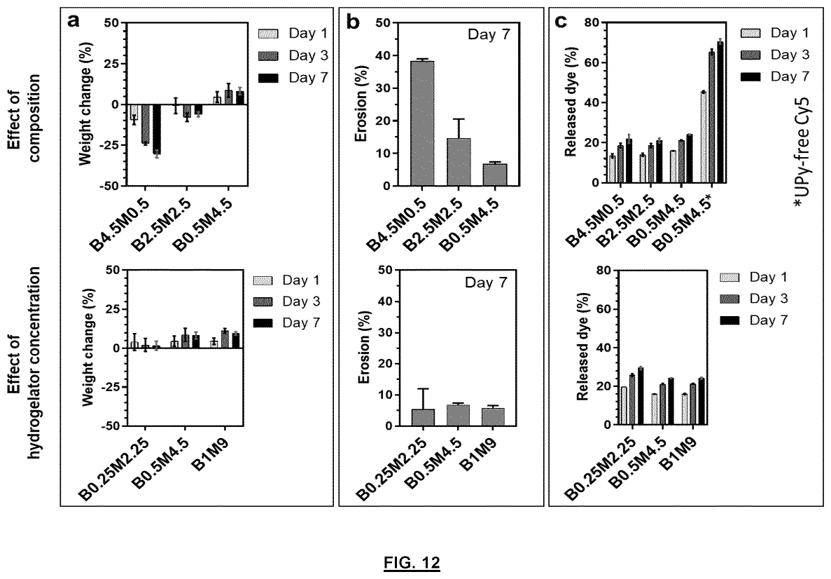

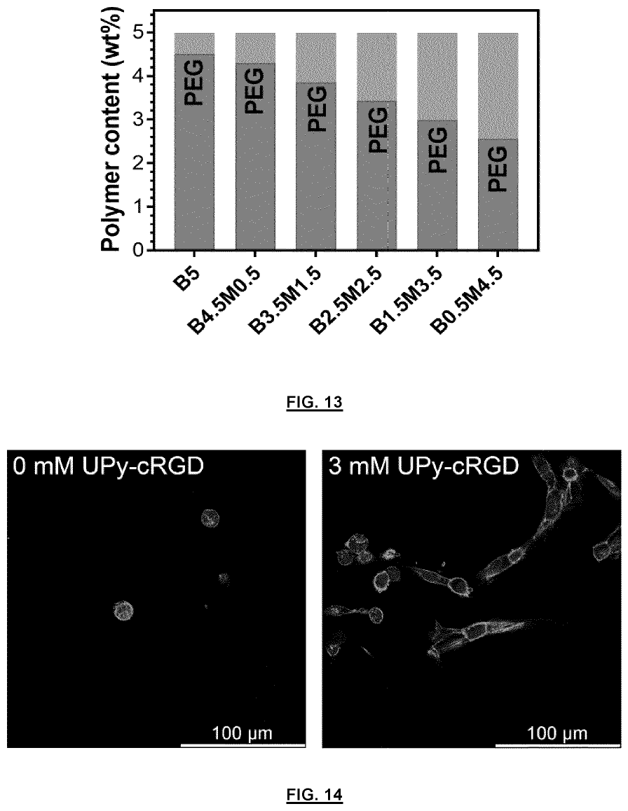

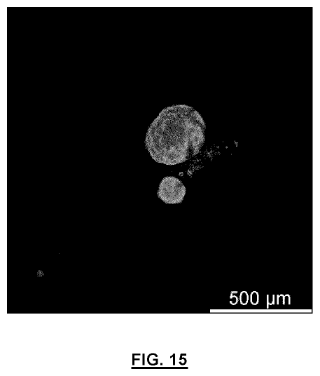

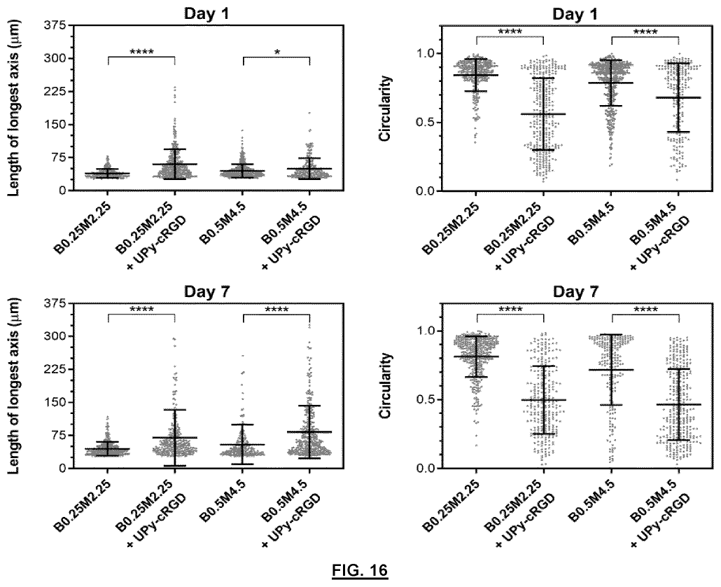

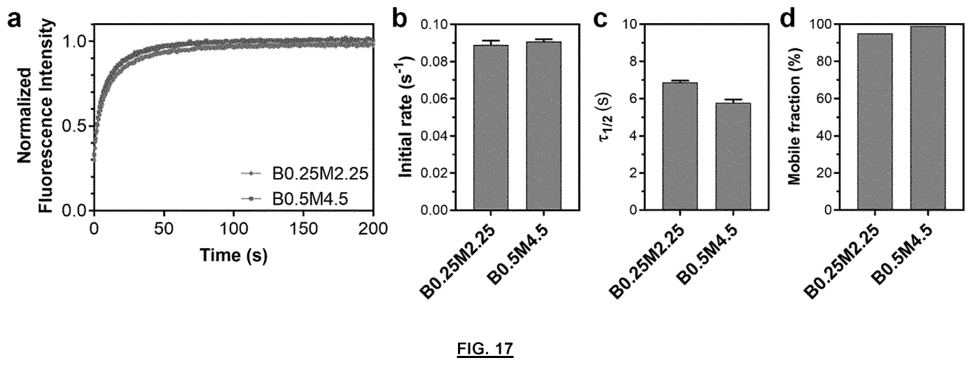

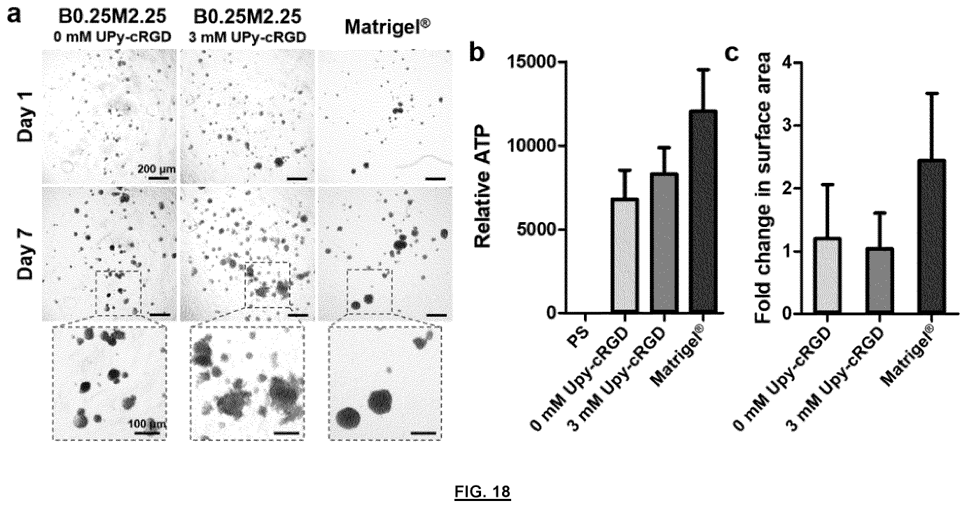

1. A method of producing a supramolecular hydrogel, wherein the supramolecular hydrogel is formed by the mixing and gelation of at least two dispersions of different types of synthetic hydrogelators, said synthetic hydrogelators being formed of synthetic building blocks comprising one or more hydrogen bonding units, wherein each bonding unit comprises a ureido-pyrimidinone subunit and each bonding unit is conjugated with a hydrophilic polymer unit, wherein the method comprises the steps of:
a) providing a first dispersion of one type of synthetic hydrogelators;
b) mixing the first dispersion of one type of synthetic hydrogelators provided in step a) with a second dispersion of another type of synthetic hydrogelators; and
c) allowing the dispersions of synthetic hydrogelators mixed in step b) to form the supramolecular hydrogel,
wherein the types of synthetic hydrogelators are selected from the group consisting of multifunctional synthetic hydrogelators comprising two or more hydrogen bonding units and monofunctional synthetic hydrogelators comprising one hydrogen bonding unit,
characterised in that the steps of the method are conducted under biocompatible conditions, and
in that the hydrophilic polymer unit of the synthetic hydrogelators comprised in the first dispersion has a minimal hydrophilicity such that the first dispersion does not form a hydrogel under the biocompatible conditions applied to the method.

2. The method according to claim 1, wherein the hydrogen bonding units of the synthetic hydrogelators comprised in the first dispersion of synthetic hydrogelators and the second dispersion of synthetic hydrogelators are identical.

3. The method according to claim 1, wherein the hydrophilic polymer unit of the synthetic hydrogelators comprised in the second dispersion has a minimal hydrophilicity such that the second dispersion does not form a hydrogel under the biocompatible conditions applied to the method.

4. The method according to claim 1, wherein the hydrogen bonding units further comprises an urea subunit and/or urethane subunit linking the ureido-pyrimidinone subunit with the hydrophilic polymer unit.

5. The method according to claim 1, wherein the hydrophilicity of the hydrophilic polymer unit is selected such that the hydrophilicity corresponds to the hydrophilicity of a monodisperse polyethylene glycol having 5 to 50 oxyethylene units.

6. The method according to claim 1, wherein, before mixing the first dispersion of synthetic hydrogelators with the second dispersion of synthetic hydrogelators in step b), the method comprises the step of adding biological material, such as cells, spheroids and/or organoids, to the first dispersion of synthetic hydrogelators.

7. The method according to claim 1, wherein, in step b), the first and second dispersions of synthetic hydrogelators are mixed such that the molar ratio between the multifunctional synthetic hydrogelators and monofunctional synthetic hydrogelators is at least 1:1, preferably at least 1:10, more preferably at least 1:50.

8. The method according to claim 1, wherein the total amount of synthetic hydrogelators in step b) is at most 25 wt.-% of the total weight of the dispersions mixed, preferably between 0.5 wt.-% and 20 wt.-%, more preferably between 1.5 wt.-% and 10 wt.-%, between 2.0 wt-% and 5 wt-%, most preferably about 2.5 wt-% or about 5.0 wt.-%.

9. The method according to claim 1, wherein the multifunctional synthetic hydrogelators are selected from the group consisting of bifunctional synthetic hydrogelators comprising two hydrogen bonding units.

10. The method according to claim 1, wherein the hydrophilic polymer unit of the monofunctional synthetic hydrogelators comprises at one end, which one end is not conjugated to the hydrogen bonding unit, a functional subunit, such as a bioactive subunit, wherein the bioactive subunit include a bioactive feature directing cell behaviour, such as cell growth, cell adhesion, cell spreading, cell migration, cell differentiation and combinations thereof and/or a bioactive feature having antimicrobial activity.

11. The method according to claim 1, wherein the method further comprises the steps of:
d) after formation of the supramolecular hydrogel, culturing biological material, such as cells, spheroids and/or organoids, for a period of time; and
e) optionally, removing the hydrogel by using external stimuli.

12. A supramolecular hydrogel obtained by the method according to claim 1.

13. The supramolecular hydrogel according to claim 12, wherein the supramolecular hydrogel is a bioactive supramolecular hydrogel.

14. A method for directing cell behavior comprising in vitro use of the supramolecular hydrogel according to any claim 12, wherein the directing of cell behaviour comprises directing one or more of as cell growth, cell adhesion, cell spreading, cell migration, cell differentiation and combinations thereof.

15. The supramolecular hydrogel according to claim 12, wherein the supramolecular hydrogel is configured for in vivo application, such as tissue or organ regeneration or therapies.

16. A kit for producing a supramolecular hydrogel, wherein the kit comprises at least two dispersions of synthetic hydrogelators, wherein the at least two dispersions of different types of synthetic hydrogelators comprise a first dispersion of one type of synthetic hydrogelators and a second dispersion of another type of synthetic hydrogelators for use in the method according to claim 1.


SEQUENCE LISTING (7 sequences)

SEQ ID NO: 1 - protein, 4 aa, Artificial Sequence
  ECM derived peptide DGEA
DGEA

SEQ ID NO: 2 - protein, 5 aa, Artificial Sequence
  ECM-derived peptides YIGSR
YIGSR

SEQ ID NO: 3 - protein, 5 aa, Artificial Sequence
  ECM-derived peptide PHSRN
PHSRN

SEQ ID NO: 4 - protein, 7 aa, Artificial Sequence
  Collagen binding peptide HVWMQAP
HVWMQAP

SEQ ID NO: 5 - protein, 9 aa, Artificial Sequence
  SDF1alpha truncated peptide SKPVVLSYR
SKPVVLSYR

SEQ ID NO: 6 - protein, 9 aa, Artificial Sequence
  SDF1alpha truncated peptide SKPVSLSYR
SKPVSLSYR

SEQ ID NO: 7 - protein, 9 aa, Artificial Sequence
  Heparin binding peptide GLRKKLGKA
GLRKKLGKA